(19) United States
(12) Patent Application Publication
(10) Pub. No.: US 2006/0073592 A1
(43) Pub. Date: Apr. 6, 2006

(54) Methods of storing tissue matrices

(76) Inventors: Wendell Sun, Warrington, PA (US); Herbert Daniel Beniker, San Antonio, TX (US); David J. McQuillan, Doylestown, PA (US)

Correspondence Address: FISH & RICHARDSON P.C., P.O. BOX 1022, MINNEAPOLIS, MN, 55440-1022, US

(21) Appl. No.: 10/959,780

(22) Filed: Oct. 6, 2004

(51) Int. Cl. C12N 5/02

(52) U.S. Cl. 435/423

(57) ABSTRACT

The invention provides methods of storing acellular tissue matrices in which a substantial portion of water in the matrices is replaced with a water-replacing agent, e.g., glycerol. Also included in the invention are compositions made by these methods as well as methods of treatment using such compositions.

### TECHNICAL FIELD

This invention relates generally to tissue matrices that can be implanted in or grafted to vertebrate subjects, and more particularly to methods of storing such tissue matrices without substantial loss of structural or functional integrity.

### BACKGROUND

Tissue matrices are increasingly being used for the repair of damaged tissues and organs or the amelioration of defective tissues and organs. A significant problem in the field has been lability of the tissue matrices and the need for relatively sophisticated equipment to store them for extended periods of time.

### SUMMARY

The inventors have found that acellular tissue matrices (ATM) in which a substantial proportion of water has been replaced with one or more water-replacing agents can be stored for extended periods of time at ambient temperature without substantial loss of structural or functional integrity. Moreover, the inventors observed that these tissue matrices showed enhanced resistance to elevated temperatures and to the deleterious effects of γ-radiation. The invention thus provides compositions containing ATM that can be stored for extended periods of time and one or more water-replacing agents, methods of making such compositions (including sterilization), and methods of treatment using the compositions.

More specifically, the invention features a composition containing: an isolated acellular tissue matrix (ATM); and within the ATM, a water-replacing reagent (WRR), the ATM containing not more than 30% of the water that the matrix contains if fully hydrated. The amount of water within the matrix can be sufficiently low to allow storage of the composition at ambient temperatures for an extended period of time without substantial damage to the ATM. The WRR can contain glycerol as the only water-replacing agent (WRA) or with other WRA. The WRR can contain one or more water-replacing agents, e.g., dimethylsulfoxide (DMSO) or polyhydroxyl compounds. The polyhydroxyl compounds can be monosaccharides, disaccharides, oligosaccharides, polysaccharides, poly-glycerol, ethylene glycol, propylene glycol, polyethylene glycol (PEG), or polyvinyl alcohols (PVA). The WRR can contain, for example, glycerol and ethylene glycol, e.g., glycerol and ethylene glycol in equal concentrations by weight, by volume, or by molarity. The ATM can include dermis from which all, or substantially all, viable cells have been removed Alternatively, the ATM can include a tissue from which all, or substantially all, viable cells have been removed, the tissue being fascia, pericardial tissue, dura, umbilical cord tissue, placental tissue, cardiac valve tissue, ligament tissue, tendon tissue, arterial tissue, venous tissue, neural connective tissue, urinary bladder tissue, ureter tissue, or intestinal tissue. The ATM can be made from human tissue or from a non-human mammalian tissue, e.g., porcine tissue or bovine tissue. The ATM can be in a non-particulate form or in a particulate form. The composition can contain, in addition, one or more supplementary agents. The supplementary agents can be, for example, radical scavengers, protein hydrolysates, tissue hydrolysates, or tissue breakdown products. Moreover, they can be tocopherols, hyaluronic acid, chondroitin sulfate, proteoglycans, monosaccharides, disaccharides, oligosaccharides, polysaccharides, sugar alcohols, and starch derivatives. Starch derivatives can be maltodextrins, hydroxyethyl starch (HES), or hydrogenated starch hydrolysates (HSH) and sugar alcohols can be adonitol, erythritol, mannitol, sorbitol, xylitol, lactitol, isomalt, maltitol, or cyclitols.

In another embodiment the invention provides a method of making a tissue matrix composition. The method includes: providing an ATM, the ATM being fully hydrated or partially dehydrated; and a process that includes sequentially exposing the whole body of the ATM to increasing concentrations of a water-replacing reagent. The process: (i) results in a composition containing a processed ATM that contains not more 30% of the water that the ATM would contain if it was fully hydrated; and (ii) does not result in substantially irreversible shrinkage of the ATM. The WRR and WRA can be any of those recited above. Where the WRR contains glycerol as the only WRA, the initial concentration of glycerol to which the ATM is exposed can be about 40% volume to volume (v/v) and the final concentration of glycerol can be about 85% v/v. The ATM can be any of those listed above. The method can further involve, after the process, heating the composition at a temperature and for a period of time sufficient to inactivate substantially all viruses in the ATM. The temperature can be, for example, 45° C. to 65° C. and the period of time can be more than 10 minutes. The method can also further involve, with or without the heating step, exposing the composition to γ, x, or e-beam radiation. The composition can be exposed such that the ATM absorbs, for example, 6 kGy to 30 kGy of the radiation. In addition, the method can involve, with or without the heating and/or irradiation step, exposing the composition to ultraviolet irradiation.

In the method, the water-replacing process can involve sequentially incubating the ATM in at least two aqueous solutions, each solution containing a higher concentration of the water-replacing reagent than the previous solution in which the ATM was incubated. The water-replacing agent contain glycerol as the only water-replacing agent and the at least two solutions can be; for example, three solutions and the concentration of glycerol: (a) in the first solution can be about 30% v/v; (b) in the second solution can be about 60% v/v; and (c) in the third solution can be about 85% v/v. Alternatively, the concentration of glycerol: (a) in the first solution can be about 40% v/v; (b) in the second solution can be about 60% v/v; and (c) in the third solution can be about 85% v/v. Moreover, the at least two solutions can be four solutions and the concentration of glycerol: (a) in the first solution can be about 40% v/v; (b) in the second solution can be about 55% v/v; (c) in the third solution can be about 70% v/v; and (d) in the fourth solution can be about 85% v/v.

Alternatively, the water-replacing process can involve exposing the matrix to a continuous increasing concentration gradient of the reagent.

In the method, the water-replacing reagent can contain one or more of the supplementary agents listed above.

Also embraced by the invention is a method of treatment. The method involves: (a) identifying a vertebrate subject as having an or organ, or tissue, in need of repair or amelioration; and (b) placing the composition in or on the organ or tissue. The method can further involve, prior to the placing, rinsing the composition in a physiological solution until the concentration of water-replacing agent in the composition is at a physiologically acceptable level. The vertebrate subject can have an abdominal wall defect or an abdominal wall injury. The organ or tissue of the vertebrate subject can be skin, bone, cartilage, meniscus, dermis, myocardium, periosteum, artery, vein, stomach, small intestine, large intestine, diaphragm, tendon, ligament, neural tissue, striated muscle, smooth muscle, bladder, urethra, ureter, gingival, or fascia (e.g., abdominal wall fascia). The gingiva can be, or can be proximal to, receding gingival. The gingiva can also include a dental extraction socket. The vertebrate subject can be a mammal, e.g., a human.

As used herein, the term “placing” a composition includes, without limitation, setting, injecting, infusing, pouring, packing, layering, spraying, and encasing the composition. In addition, placing “on” a recipient tissue or organ means placing in a touching relationship with the recipient tissue or organ.

As used herein, the term “operably linked” means incorporated into a genetic construct so that expression control sequences (i.e., transcriptional and translational regulatory elements) effectively control expression of a coding sequence of interest. Transcriptional and translational regulatory elements include but are not limited to inducible and non-inducible promoters, enhancers, operators and other elements that are known to those skilled in the art and that drive or otherwise regulate gene expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention, e.g., ATM compositions that can be stored for extended periods of time at ambient temperatures, will be apparent from the following description, from the drawings and from the claims.

### DESCRIPTION OF DRAWINGS

FIGS. 1A and B are line graphs showing the relative amount of glycerol in two acellular dermal matrices (ADM) with thicknesses of approximately 1.6 mm (FIG. 1A) and approximately 3.0 mm (FIG. 1B) after sequential incubations for various lengths of time in three solutions containing 40% (volume to volume; v/v), 60% v/v, and 85% v/v glycerol.

FIG. 2 is a line graph showing the decrease in the amount of glycerol and the increase in the amount of water in a water-replaced (with glycerol) ADM after incubation for various lengths of time in normal saline. Data are mean ± standard deviation of three replicates.

FIGS. 3A and B are photomicrographs of an ADM that had been subjected to water replacement followed by rehydration (FIG. 3B; “Preserved, rehydrated tissue”) and a control ADM that had been prepared in the same way as that shown in FIG. 3B but had not been subjected to water replacement and rehydration (FIG. 3A; “Control tissue”).

FIGS. 4A and 4B are two photomicrographs showing an ADM that underwent water replacement (with glycerol) and was then irradiated with 24 kGy of γ-radiation (FIG. 4B; “γ-irradiated (24 kGy)”) and a control ADM that underwent the same water replacement procedure (with glycerol) but was not irradiated (FIG. 4A; “Control tissue”).

FIG. 5 is a photomicrograph of an ADM that had sequentially: (a) undergone water replacement with glycerol; (b) been stored in the water-replaced state for four days at room temperature; (c) been rehydrated; (d) been implanted into a nude mouse; and (e) 21 days after implantation been removed from the nude mouse and subjected to histological analysis.

FIG. 6A is a differential scanning calorimetry (DSC) thermogram of a water-replaced (with glycerol) ADM.

FIG. 6B is a line graph showing the increase in protein melting temperature in proportion to the amount of glycerol in ADM.

FIG. 7 is a photomicrograph of an ADM that had sequentially: (a) undergone water replacement with glycerol; (b) been stored in the water-replaced state for four days at between 52° C. and 59° C. (average 55° C.); (c) been rehydrated; (d) been implanted into a nude mouse; and (e) 21 days after implantation been removed from the nude mouse and subjected to histological analysis.

FIG. 8A is a line graph showing the relative amount of glycerol in acellular vein matrices (AVM) after sequential incubations for various lengths of time in two solutions containing 50% (volume to volume; v/v) and 90% v/v ethylene glycol (EG). Data are mean ± standard deviation of three replicates.

FIG. 8B is a line graph showing the decrease in the amount of EG and the increase in the amount of water in a water-replaced (with EG) AVM after incubation for various lengths of time in normal saline. Data are as indicated for FIG. 8A.

FIG. 9A is a line graph showing the relative amount of glycerol in AVM after sequential incubations for various lengths of time in four solutions containing 40% v/v, 55% v/v, 70% v/v, and 85% v/v glycerol. Data are as indicated for FIG. 8A.

FIG. 9B is a line graph showing the decrease in the amount of glycerol and the increase in the amount of water in a water-replaced (with glycerol) AVM after incubation for various lengths of time in normal saline. Data are as indicated for FIG. 8A.

FIG. 10 is a series of three photomicrographs of AVM that were subjected to three different water replacement procedures, rehydrated, and then subjected to histological analysis. The locations of Wharton's jelly and basement membrane in two of the photomicrographs are indicated.

### DETAILED DESCRIPTION

Various embodiments of the invention are described below.

Methods And Compositions For Storing Acellular Tissue Matrices

The methods of the invention involve removing a substantial proportion of the water from an ATM by replacing the water with one or more water-replacing agents (WRA). These WRA-containing ATM can be stored for extended periods of time under ambient temperatures. ATM that has been subjected to this water-replacing process are sometimes referred to herein as “water-replaced ATM”.

As used herein, an ATM, in which a “substantial proportion of water” has been removed, contains not more than 30% (e.g., not more than: 28%; 26%; 24%; 22%; 20%; 16%; 12%; 8%; 6%; 4%; 2%; or 1%) of the water that the relevant ATM contains when fully hydrated. As used herein, a “fully hydrated ATM” is an ATM containing the maximum amount of bound and unbound water that it is possible for that ATM to contain under atmospheric pressure. In comparing the amounts of water (unbound and/or bound) in two (or more) ATM that are fully hydrated, since the maximum amount of water than an ATM made from any particular tissue will vary with the temperature of the ATM, it is of course important that measurements for the two (or more) ATM be made at the same temperature. Examples of fully hydrated ATM include, without limitation, those at the end of the decellularizing process described in Example 1 and an ATM that has been rehydrated at room temperature (i.e., about 15° C. to about 35° C.) in 0.9% sodium chloride solution for 4 hours following a prior freeze-drying process such as those described herein. Bound water in an ATM is the water in the ATM whose molecular mobility (rotational and translational) is reduced (compared to pure bulky) due to molecular interactions (e.g., hydrogen bonding) between the water and ATM molecules and/or other phenomena (e.g., surface tension and geometric restriction) that limit the mobility of the water in the ATM. Unbound water within the ATM has the same molecular mobility properties as bulky water in dilute aqueous solutions such as, for example, biological fluids. As used herein, a “partially hydrated ATM” is an ATM that contains, at atmospheric pressure, less than but more than 30% (e.g., more than: 35%; 40%; 45%; 50%; 55%; 60%; 65%; 70%; 75%; 80%; 85%; 90%; 95%; 97%; 98%; or 99%) of the unbound and/or bound water that the same ATM would contain at atmospheric pressure when fully hydrated; again measurements of water amounts in the partially hydrated and fully hydrated ATM must be made at the same temperature.

As used herein, the term “ambient temperatures” means temperatures between −40° C. to 50° C. (e.g., −35° C. to 50° C.; −30° C. to 45° C.; −20° C. to 40° C.; −10° C. to 35° C.; 0° C. to 30° C.; −40° C. to −30° C.; −40° C. to −20° C.; −40° C. to −10° C.; −40° C. to −0° C.; −40° C. to 10° C.; −30° C. to −20° C.; −30° C. to −10° C.; −30° C. to 0° C.; −30° C. to 10° C.; −20° C. to −10° C.; −20° C. to 0° C.; −20° C. to 10° C.; −10° C. to 0° C.; −10° C. to 10° C.; 4° C. to 10° C.; 4° C. to 15° C.; 4° C. to 25° C.; 4° C. to 30° C.; 10° C. to 15° C.; 10° C. to 20° C.; 10° C. to 25° C.; 10° C. to 30° C.; 10° C. to 35° C.; 15° C. to 20° C.; 15° C. to 25° C.; 15° C. to 30° C.; 15° C. to 23° C.; 20° C. to 25° C.; 20° C. to 25° C.; 20° C. to 30° C.; 20° C. to 35° C.; 25° C. to 30° C.; or 25° C. to 35° C.). As used herein, the term “extended period of time” means a period of time greater than two days (e.g., greater than: three days; four days; five days; six days; seven days; eight days; nine days; 10 days; 11 days; 12 days; 13 days; two weeks; three weeks; one month; two months; three months; four months; five months; six months; seven months; eight months; nine months; 10 months; 11 months; 12 months; 15 months; 18 months; 22 months; 2 years; 2.5 years; 3 years; 3.5 years; 4 years; 5 years; or 6 years).

As used herein the term “substantial damage” to an ATM means an increase in the level of collagen damage in the ATM by more than 25% in the ATM. Thus, as used herein, any process (e.g., water removal and/or storage after water removal), agent, or composition that does not cause “substantial damage” to an ATM is a process, agent, or composition that does not increase the level of collagen damage in the ATM by more than 25% of the collagen damage existing in the ATM prior to performance of the process or exposure of the ATM to the agent or composition. “Collagen damage” is described in Example 8.

ATM

As used herein, an “acellular tissue matrix” (“ATM”) is a tissue-derived structure that is made from any of a wide range of collagen-containing tissues by removing all, or substantially all, viable cells and all detectable subcellular components and/or debris generated by killing cells. As used herein, an ATM lacking “substantially all viable cells” is an ATM in which the concentration of viable cells is less than 1% (e.g., less than: 0.1%; 0.01%; 0.001%; 0.0001%; 0.00001%; or 0.000001%) of that in the tissue or organ from which the ATM was made.

The ATM of the invention preferably, but not necessarily, lack, or substantially lack, an epithelial basement membrane. The epithelial basement membrane is a thin sheet of extracellular material contiguous with the basilar aspect of epithelial cells. Sheets of aggregated epithelial cells form an epithelium. Thus, for example, the epithelium of skin is called the epidermis, and the skin epithelial basement membrane lies between the epidermis and the dermis. The epithelial basement membrane is a specialized extracellular matrix that provides a barrier function and an attachment surface for epithelial-like cells; however, it does not contribute any significant structural or biomechanical role to the underlying tissue (e.g., dermis). Unique components of epithelial basement membranes include, for example, laminin, collagen type VII, and nidogen. The unique temporal and spatial organization of the epithelial basement membrane distinguish it from, e.g., the dermal extracellular matrix. The presence of the epithelial basement membrane in an ATM of the invention could be disadvantageous in that the epithelial basement membrane likely contains a variety of species-specific components that would elicit the production of antibodies, and/or bind to preformed antibodies, in xenogeneic graft recipients of the acellular matrix. In addition, the epithelial basement membrane can act as barrier to diffusion of cells and/or soluble factors (e.g., chemoattractants) and to cell infiltration. Its presence in ATM grafts can thus significantly delay formation of new tissue from the acellular tissue matrix in a recipient animal. As used herein, an ATM that “substantially lacks” an epithelial basement membrane is an acellular tissue matrix containing less than 5% (e.g., less than: 3%; 2%; 1%; 0.5%; 0.25%; 0.1%; 0.01%; 0.001%; or even less than 0.001%) of the epithelial basement membrane possessed by the corresponding unprocessed tissue from which the acellular tissue matrix was derived.

Biological functions retained by ATM include cell recognition and cell binding as well as the ability to support cell spreading, cell proliferation, and cell differentiation. Such functions are provided by undenatured collagenous proteins (e.g., type I collagen) and a variety of non-collagenous molecules (e.g., proteins that serve as ligands for either molecules such as integrin receptors, molecules with high charge density such glycosaminoglycans (e.g., hyaluronan) or proteoglycans, or other adhesins). Structural functions retained by useful acellular matrices include maintenance of histological architecture, maintenance of the three-dimensional array of the tissue's components and physical characteristics such as strength, elasticity, and durability, defined porosity, and retention of macromolecules. The efficiency of the biological functions of an ATM can be measured, for example, by the ability of the ATM to support cell proliferation and is at least 50% (e.g., at least: 50%; 60%; 70%; 80%; 90%; 95%; 98%; 99%; 99.5%; 100%; or more than 100%) of that of the native tissue or organ from which the ATM is made.

It is not necessary that the grafted matrix material be made from tissue that is identical to the surrounding host tissue but should simply be amenable to being remodeled by invading or infiltrating cells such as differentiated cells of the relevant host tissue, stem cells such as mesenchymal stem cells, or progenitor cells. Remodelling is directed by the above-described ATM components and signals from the surrounding host tissue (such as cytokines, extracellular matrix components, biomechanical stimuli, and bioelectrical stimuli). The presence of mesenchymal stem cells in the bone marrow and the peripheral circulation has been documented in the literature and shown to regenerate a variety of musculoskeletal tissues [Caplan (1991) J. Orthop. Res. 9:641-650; Caplan (1994) Clin. Plast. Surg. 21:429-435; and Caplan et al. (1997) Clin Orthop. 342:254-269]. Additionally, the graft must provide some degree (greater than threshold) of tensile and biomechanical strength during the remodeling process.

It is understood that the ATM can be produced from any collagen-containing soft tissue and muscular skeleton (e.g., dermis, fascia, pericardium, dura, umbilical cords, placentae, cardiac valves, ligaments, tendons, vascular tissue (arteries and veins such as saphenous veins), neural connective tissue, urinary bladder tissue, ureter tissue, or intestinal tissue), as long as the above-described properties are retained by the matrix. Moreover, the tissues in which the above allografts are placed include essentially any tissue that can be remodeled by invading or infiltrating cells. Relevant tissues include, without limitation, skeletal tissues such as bone, cartilage, ligaments, fascia, and tendon. Other tissues in which any of the above allografts can be placed include, without limitation, skin, gingiva, dura, myocardium, vascular tissue, neural tissue, striated muscle, smooth muscle, bladder wall, ureter tissue, intestine, and urethra tissue.

Furthermore, while an ATM will generally have been made from one or more individuals of the same species as the recipient of the ATM graft, this is not necessarily the case. Thus, for example, an ATM can have been made from a porcine tissue and be implanted in a human patient. Species that can serve as recipients of ATM and donors of tissues or organs for the production of the ATM include, without limitation, humans, no-human primates (e.g., monkeys, baboons, or chimpanzees), porcine, bovine, horses, goats, sheep, dogs, cats, rabbits, guinea pigs, gerbils, hamsters, rats, or mice. Of particular interest as donors are animals (e.g., pigs) that have been genetically engineered to lack the terminal galactose-α1-3 galactose moiety. For descriptions of appropriate animals see co-pending U.S. application Ser. No. 10/896,594 and U.S. Pat. No. 6,166,288, the disclosures of all of which are incorporated herein by reference in their entirety.

The form in which the ATM is provided will depend on the tissue or organ from which it is derived and on the nature of the recipient tissue or organ, as well as the nature of the damage or defect in the recipient tissue or organ. Thus, for example, a matrix derived from a heart valve can be provided as a whole valve, as small sheets or strips, as pieces cut into any of a variety of shapes and/or sizes, or in a particulate form. The same concept applies to ATM produced from any of the above-listed tissues and organs. It is understood that an ATM useful for the invention can be made from a recipients own collagen-based tissue.

The ATM can be produced by any of a variety of methods. All that is required is that the steps used in their production result in matrices with the above-described biological and structural properties. Particularly useful methods of production include those described in U.S. Pat. Nos. 4,865,871 and 5,366,616 and copending U.S. application Ser. Nos. 09/762,174, 10/165,790, and 10/896,594, all of which are incorporated herein by reference in their entirety.

In brief, the steps involved in the production of an ATM generally include harvesting the tissue from a donor (e.g., a human cadaver or any of the above-listed mammals), chemical treatment so as to stabilize the tissue and avoid biochemical and structural degradation together with or followed by cell removal under conditions which similarly preserve biological and structural function. After thorough removal of dead and/or lysed cell components that may cause inflammation as well any bioincompatible cell-removal agents, the matrix can be subjected to the water-replacement method of the invention (see below). Alternatively, the ATM can be treated with a cryopreservation agent and cryopreserved and, optionally, freeze dried, again under conditions necessary to maintain the described biological and structural properties of the matrix. After freeze drying, the tissue can, optionally, be pulverized or micronized to produce a particulate ATM under similar function-preserving conditions. After cryopreservation or freeze-drying (and optionally pulverization or micronization), the ATM can be thawed or rehydrated, respectively, and then subjected to the water-replacement method of the invention (see below). All steps are generally carried out under aseptic, preferably sterile, conditions.

The initial stabilizing solution arrests and prevents osmotic, hypoxic, autolytic, and proteolytic degradation, protects against microbial contamination, and reduces mechanical damage that can occur with tissues that contain, for example, smooth muscle components (e.g., blood vessels). The stabilizing solution generally contains an appropriate buffer, one or more antioxidants, one or more oncotic agents, one or more antibiotics, one or more protease inhibitors, and in some cases, a smooth muscle relaxant.

The tissue is then placed in a processing solution to remove viable cells (e.g., epithelial cells, endothelial cells, smooth muscle cells, and fibroblasts) from the structural matrix without damaging the basement membrane complex or the biological and structural integrity of the collagen matrix. The processing solution generally contains an appropriate buffer, salt, an antibiotic, one or more detergents, one or more agents to prevent cross-linking, one or more protease inhibitors, and/or one or more enzymes. Treatment of the tissue must be (a) with a processing solution containing active agents at a concentration and (b) for a time period such that the structural integrity of the matrix is maintained.

After the tissue is decellularized, it can be subjected to the water replacement method of the invention (see below).

Alternatively, the tissue can be cryopreserved prior to undergoing water replacement. If so, after decellularization, the tissue is incubated in a cryopreservation solution. This solution generally contains one or more cryoprotectants to minimize ice crystal damage to the structural matrix that could occur during freezing. If the tissue is to be freeze dried, the solution will generally also contain one or more dry-protective components, to minimize structural damage during drying and may include a combination of an organic solvent and water which undergoes neither expansion or contraction during freezing. The cryoprotective and dry-protective agents can be the same one or more substances. If the tissue is not going to be freeze dried, it can be frozen by placing it (in a sterilized container) in a freezer at about −80° C., or by plunging it into sterile liquid nitrogen, and then storing at a temperature below −160° C. until use. The sample can be thawed prior to use by, for example, immersing a sterile non-permeable vessel (see below) containing in a water bath at about 37° C. or by allowing the tissue to come to room temperature under ambient conditions.

If the tissue is to be frozen and freeze dried, following incubation in the cryopreservation solution, the tissue is packaged inside a sterile vessel that is permeable to water vapor yet impermeable to bacteria, e.g., a water vapor permeable pouch or glass vial. One side of a preferred pouch consists of medical grade porous Tyvek® membrane, a trademarked product of DuPont Company of Wilmington, Del. This membrane is porous to water vapor and impervious to bacteria and dust. The Tyvek membrane is heat sealed to a impermeable polythylene laminate sheet, leaving one side open, thus forming a two-sided pouch. The open pouch is sterilized by irradiation (e.g., γ-irradiation) prior to use. The tissue is aseptically placed (through the open side) into the sterile pouch. The open side is then aseptically heat sealed to close the pouch. The packaged tissue is henceforth protected from microbial contamination throughout subsequent processing steps.

The vessel containing the tissue is cooled to a low temperature at a specified rate which is compatible with the specific cryoprotectant formulation to minimize the freezing damage. See U.S. Pat. No. 5,336,616 for examples of appropriate cooling protocols. The tissue is then dried at a low temperature under vacuum conditions, such that water vapor is removed sequentially from each ice crystal phase.

At the completion of the drying of the samples in the water vapor permeable vessel, the vacuum of the freeze drying apparatus is reversed with a dry inert gas such as nitrogen, helium or argon. While being maintained in the same gaseous environment, the semipermeable vessel is placed inside an impervious (i.e., impermeable to water vapor as well as microorganisms) vessel (e.g., a pouch) which is further sealed, e.g., by heat and/or pressure. Where the tissue sample was frozen and dried in a glass vial, the vial is sealed under vacuum with an appropriate inert stopper and the vacuum of the drying apparatus reversed with an inert gas prior to unloading. In either case, the final product is hermetically sealed in an inert gaseous atmosphere.

The freeze dried tissue may be stored under refrigerated conditions until being submitted to the water-replacement process (see below).

After rehydration of water-replaced ATM (see below), histocompatible, viable cells can be restored to the ATM to produce a permanently accepted graft that may be remodeled by the host. This is generally done just prior to placing of the ATM in a mammalian subject. Where the matrix has been freeze dried, it will be done after rehydration. In a preferred embodiment, histocompatible viable cells may be added to the matrices by standard in vitro cell coculturing techniques prior to transplantation, or by in vivo repopulation following transplantation. In vivo repopulation can be by the recipient's own cells migrating into the ATM or by infusing or injecting cells obtained from the recipient or histocompatible cells from another donor into the ATM in situ.

The cell types used for reconstitution will depend on the nature of the tissue or organ to which the ATM is being remodelled. For example, the primary requirement for reconstitution of full-thickness skin with an ATM is the restoration of epidermal cells or keratinocytes. For example, cells derived directly from the intended recipient can be used to reconstitute an ATM and the resulting composition grafted to the recipient in the form of a meshed split-skin graft. Alternatively, cultured (autologous or allogeneic) cells can be added to ATM. Such cells can be, for example, grown under standard tissue culture conditions and then added to the ATM. In another embodiment, the cells can be grown in and/or on an ATM in tissue culture. Cells grown in and/or on an ATM in tissue culture can have been obtained directly from an appropriate donor (e.g., the intended recipient or an allogeneic donor) or they can have been first grown in tissue culture in the absence of the ATM.

The most important cell for reconstitution of heart valves and vascular conduits is the endothelial cell, which lines the inner surface of the tissue. Endothelial cells may also be expanded in culture, and may be derived directly from the intended recipient patient or from umbilical arteries or veins.

Other cells with which the matrices can be repopulated include, but are not limited to, fibroblasts, embryonic stem cells (ESC), adult or embryonic mesenchymal stem cells (MSC), prochondroblasts, chondroblasts, chondrocytes, pro-osteoblasts, osteocytes, osteoclasts, monocytes, pro-cardiomyoblasts, pericytes, cardiomyoblasts, cardiomyocytes, gingival epithelial cells, or periodontal ligament stem cells. Naturally, the ATM can be repopulated with combinations of two more (e.g., two, three, four, five, six, seven, eight, nine, or ten) of these cell-types.

Reagents and methods for carrying out all the above steps are known in the art. Suitable reagents and methods are described in, for example, U.S. Pat. No 5,336,616.

Particulate ATM can be made from any of the above described non-particulate ATM by any process that results in the preservation of the biological and structural functions described above and, in particular, damage to collagen fibers, including sheared fiber ends, should be minimized. Many known wetting and drying processes for making particulate ATM do not so preserve the structural integrity of collagen fibers.

One appropriate method for making particulate ATM is described in U.S. patent application Ser. No.09/762,174. The process is briefly described below with respect to a freeze dried dermal ATM but one of skill in the art could readily adapt the method for use with freeze dried ATM derived from any of the other tissues listed herein.

The acellular dermal matrix can be cut into strips (using, for example, a Zimmer mesher fitted with a non-interrupting “continuous” cutting wheel). The resulting long strips are then cut into lengths of about 1 cm to about 2 cm. A homogenizer and sterilized homogenizer probe (e.g., a LabTeck Macro homogenizer available from OMNI International, Warrenton, Va.) is assembled and cooled to cryogenic temperatures (i.e., about <196° C. to about −160° C.) using sterile liquid nitrogen which is poured into the homogenizer tower. Once the homogenizer has reached a cryogenic temperature, cut pieces of ATM are added to the homogenizing tower containing the liquid nitrogen. The homogenizer is then activated so as to cryogenically fracture the pieces of ATM. The time and duration of the cryogenic fracturing step will depend upon the homogenizer utilized, the size of the homogenizing chamber, and the speed and time at which the homogenizer is operated, and are readily determinable by one skilled in the art. As an alternative, the cryofracturing process can be conducted in cryomill cooled to a cryogenic temperature.

The cryofractured particulate acellular tissue matrix is, optionally, sorted by particle size by washing the product of the homogenization with sterile liquid nitrogen through a series of metal screens that have also been cooled to a cryogenic temperature. It is generally useful to eliminate large undesired particles with a screen with a relatively large pore size before proceeding to one (or more screens) with a smaller pore size. Once isolated, the particles can be freeze dried to ensure that any residual moisture that may have been absorbed during the procedure is removed. The final product is a powder (usually white or off-white) generally having a particle size of about 1 micron to about 900 microns, about 30 microns to about 750 microns, or about 150 to about 300 microns. The material is readily rehydrated by suspension in normal saline or any other suitable rehydrating agent known in the art. It may also be suspended in any suitable carrier known in the art (see, for example, U.S. Pat. No. 5,284,655 incorporated herein by reference in its entirety). If suspended at a high concentration (e.g., at about 600 mg/ml), the particulate ATM can form a “putty”, and if suspended at a somewhat lower concentration (e.g., about 330 mg/ml), it can form a “paste”. Such putties and pastes can conveniently be packed into, for example, holes, gaps, or spaces of any shape in tissues and organs so as to substantially fill such holes, gaps, or spaces.

One highly suitable freeze dried ATM is produced from human dermis by the LifeCell Corporation (Branchburg, N.J.) and marketed in the form of small sheets as AlloDerm®. Such sheets are marketed by the LifeCell Corporation as rectangular sheets with the dimensions of, for example, 1 cm×2 cm, 3 cm×7 cm, 4 cm×8 cm, 5 cm×10 cm, 4 cm×12 cm, and 6 cm×12 cm. The cryoprotectant used for freezing and drying Alloderm is a solution of 35% maltodextrin and 10mM ethylenediaminetetraacetate (EDTA). Thus, the final dried product contains about 60% by weight ATM and about 40% by weight maltodextrin. The LifeCell Corporation also makes an analogous product made from porcine dermis (designated XenoDerm) having the same proportions of ATM and maltodextrin as AlloDerm. In addition, the LifeCell Corporation markets a particulate acellular dermal matrix made by cryofracturing AlloDerm (as described above) under the name Cymetra®. The particle size for Cymetra is in the range of about 60 microns to about 150 microns as determined by mass.

The particles of particulate or pulverized (powdered) ATM of the invention will be less than 1.0 mm in their longest dimension. Pieces of ATM with dimensions greater than this are non-particulate acellular matrices.

WRA

As used herein, the term “water-replacing agent” (“WRA”) refers to chemical compounds that substitute for water and (a) provide similar hydrogen-bonding for structural and consequent function preservation of the ATM; but (b) lack, or substantially lack, the properties of water (e.g., reactive or catalytic properties) that result in substantial damage to ATM. An agent or composition that “substantially lacks” these properties of water is an agent or composition that causes no more than 30% of the damage caused by water under the same conditions (temperature and time) of exposure. As used herein, the term “water-replacing reagent” (“WRR”) refers to a single WRA or a mixture of two or more (e.g., three, four, five, six, seven, eight, nine, ten, 11, 12, 15, 20, or more) WRA.

WRA useful for the invention include any of a variety of compounds with the properties described above and are well known in the art. They include compounds such as dimethylsulfoxide (DMSO), sodium glycerophosphate and any of a wide range of polyhydroxyl compounds (also sometimes called polyhydroxy or polyol compounds) such as many carbohydrates (e.g., monosaccharides, disaccharides, oligosaccharides, and polysaccharides), sugar alcohols (see examples below), glycerol, poly-glycerol, ethylene glycol, propylene glycol, polyethylene glycol (PEG), and polyvinyl alcohols. Also useful as WRA are esters of these polyhydroxyl compounds. Other polyhydroxyl compounds (and ester derivatives thereof) useful as WRA for the invention include those listed in U.S. Pat. No. 5,284,655, the disclosure of which is incorporated herein by reference in its entirety.

The WRA can be liquids or solids at room temperature and will generally be used diluted in an aqueous solvent such as water, normal saline, phosphate buffered saline (PBS), Ringer's lactate, or a standard tissue culture medium. The WRA can be used singly or in combinations of two or more (see definition of WRR above).

The solutions containing the WRR can contain any of a variety of supplementary agents that serve to prevent or minimize the damage that can occur to ATM (see Example 8) during, for example, storage and/or sterilization procedures by any of a variety of mechanisms. Supplementary agents include, for example, free radical scavengers, tissue hydrolysates, and tissue breakdown products and any of the agents listed below as components of rehydration solutions. Compounds useful as supplementary agents include, e.g., monosaccharides, disaccharides, oligosaccharides, polysaccharides, sugar alcohols (such as adonitol, erythritol, mannitol, sorbitol, xylitol, lactitol, isomalt, maltilol, and cyclitols), starch derivatives, hyaluronic acid, and chondroitin sulfate. Starch derivatives can be, for example, maltodextrins, hydroxyethyl starch (HES), or hydrogenated starch hydrolysates (HSH).

It will be clear from the above description that that certain compounds (e.g., sugar alcohols) can function as WRA and/or as supplementary agents.

The Water-Replacement Process

ATM can be submitted to the water-replacement process of the invention immediately after procurement if made from a naturally acellular tissue or immediately after decellularization if made from cellular tissue. Alternatively, if the ATM are to undergo the water-replacement process after being cryopreserved (or freeze-dried) and then stored, frozen ATM are thawed and freeze-dried ATM are rehydrated using standard procedures. Frozen ATM can be thawed by, for example, immersing a sterile non-permeable vessel containing the ATM in a water bath at about 37° C. or by allowing the frozen ATM to come to room temperature under ambient conditions.

With respect to freeze-dried ATM, it is important to minimize osmotic forces and surface tension effects during rehydration. The aim in rehydration is to augment the selective preservation of the extracellular support matrix. Appropriate rehydration may be accomplished by, for example, an initial incubation of the dried tissue in an environment of about 100% relative humidity, followed by immersion in a suitable rehydration solution. Alternatively, the dried tissue may be directly immersed in the rehydration solution, without prior incubation, in a high humidity environment. Rehydration should not cause osmotic damage to the sample. Vapor rehydration should ideally achieve a residual moisture level of at least 15% and fluid rehydration should result in a tissue moisture level of between 20% and 70%. Depending on the tissue to be rehydrated, the rehydration solution can be, for example, normal saline, PBS, Ringer's lactate, or a standard cell culture medium. Where the ATM is subject to the action of endogenous collagenases, elastases or residual autolytic activity from previously removed cells, additives to the rehydration solution are made and include protease inhibitors. Where residual free radical activity is present, agents to protect against free radicals are used including antioxidants, and enzymatic agents that protect against free radical damage. Antibiotics may also be included to inhibit bacterial contamination. Oncotic agents being in the form of proteoglycans, dextran and/or amino acids may also be included to prevent osmotic damage to the matrix during rehydration. Rehydration of a dry sample is especially suited to this process as it allows rapid and uniform distribution of the components of the rehydration solution. In addition, the rehydration solutions may contain specific components, for example, diphosphonates to inhibit alkaline phosphatase and prevent subsequent calcification. Agents may also be included in the rehydration solution to stimulate neovascularization and host cell infiltration following transplantation of the rehydrated extracellular matrix.

The water removal process involves exposing the whole body of a fully hydrated or partially hydrated ATM to increasing concentrations of a WRR solution (see above). The process can involve either serially moving the ATM to separate WRR solutions containing increasing concentrations of the WRR. In this method, the ATM is immersed in two or more (e.g., three, four, five, six, seven, eight, nine, ten, 11, 12, or even more) WRR solutions. Alternatively, the ATM can be kept in a single vessel and exposed to a continuous and increasing concentration gradient of the WRR. Methods of generating continuous concentration gradients are known in the art. The concentration increase in any continuous gradient-based methodology can be readily achieved with, for example, synchronizing peristaltic pumps and mixers.

Where a particulate ATM is subjected to the water replacement process, it may be necessary to sediment the particles between exposure to separate solutions. This can be done by any appropriate method known in the art, e.g., filtration or centrifugation. Alternatively, a particulate ATM can be incubated in a WRR solution of low concentration, and the concentration of WRR solution can be sequentially increased without separating the ATM from the WRR solution but by sequentially adding appropriate amounts of the WRR to the solution.

Variables such as starting concentration of WRR, intermediate concentrations of WRR, the number of intermediate concentrations of WRR, final concentrations of WRR, times of incubation at each concentration of WRR, the rate of WRR concentration increase when using WRR concentration gradients, and the temperature at which the incubations are performed will vary greatly depending, for example, on the nature of the tissue from which the ATM of interest was made and the volume of the ATM. For example, tendon is a very dense tissue and longer incubations will be required in order for the WRR to reach an equilibrium concentration within ATM made from it. On the other hand, placental and venous tissue (e.g., umbilical vein tissue) have very little dry tissue mass and much shorter incubations in WRR solutions are required. Generally, incubations will be for the time necessary for the concentration of the WRR within the ATM to reach an apparent equilibrium level. Moreover, in ATM made from dense tissues, the maximum concentration of WRR achievable within the ATM is lower than for less dense tissues. Methods for establishing a workable protocol for any particular tissue are well within the expertise of, and would involve no more than routine experimentation by, those skilled in the art. Applicable experimentation can be that described herein or obvious adaptations of it. A useful protocol is one in which: (a) the amount of water in an ATM is decreased to no more than 30% of that of the ATM when fully hydrated and sufficiently low that the ATM can be stored for an extended period of time under ambient conditions; and (b) any shrinkage that the ATM undergoes during the water-replacement process is substantially reversible upon subsequent rehydration prior to grafting to, or implantation, in an appropriate recipient. As used herein, ATM shrinkage that is “substantially reversible” is shrinkage that is reversed such that the water-replaced ATM after rehydration has a volume that is at least 70% (e.g., at least: 75%; 80%; 85%; 90%; 95%; 98%; or 99%, or even 100%) of the ATM prior to the water replacement process. Naturally, while the less shrinkage that occurs during the water replacement process the better, the relevant parameter is the reversibility of any shrinkage that does occur.

When glycerol alone (as a WRR) dissolved in an appropriate aqueous solvent (e.g., normal saline) is used to process a dermal ATM, suitable starting concentrations of glycerol are 20% volume to volume (v/v) to 40% (v/v) (e.g., 25% v/v, 30% v/v, 35% v/v, 37% v/v, or 39% v/v). Suitable final concentrations of glycerol for such an ATM can be 65% v/v to 98% v/v (e.g., 68% v/v, 70% v/v, 72% v/v, 74% v/v, 76% v/v, 78% v/v, 80% v/v, 82% v/v, 84% v/v, 86% v/v, 88% v/v, 90% v/v, 92% v/v, 94% v/v, or 96% v/v). In addition the ATM can be immersed in one or two intermediate concentrations of glycerol. Such intermediate concentrations of glycerol can be, for example, 45% v/v, 50% v/v, 55% v/v, 60% v/v, 65% v/v, 70% v/v, 75% or 80% v/v. Incubations at lower concentrations of glycerol (e.g., 30% v/v) can be for 20 minutes to 2 hours and at higher concentrations (e.g., concentrations greater than 60% v/v) can be for 1 to 4 hours. As used herein, the term “about”, when applied to v/v concentrations of glycerol used as a WRA, indicates that the concentration of glycerol can vary by up to three percentage points from the stated percentage. Thus, for example, the concentration of glycerol in a solution containing “about 70% v/v” glycerol can contain between 67% v/v and 73% v/v glycerol.

At the end of the process, the resulting water-replaced ATM can be stored at ambient temperature for an extended period of time (see above). Alternatively, it can be stored refrigerated, e.g., in liquid N₂ or at −80° C., −50° C., −20° C., −10° C., 0° C., 4° C., or 10° C.

Optionally, the water-replaced ATM can be submitted to treatments to diminish their bioburden For example they can be exposed to elevated temperatures (e.g., 45° C. to 65° C: e.g., 48° C., 50° C., 53° C., 55° C., 56° C., 58° C.,60° C., 62° C., 63° C., or 64° C.) for a suitable period of time. Times of exposure can be 15 minutes to several days or weeks, e.g., 20 minutes, 30 minutes, 45 minutes, one hour, two hours, five hours, eight hours, 12 hours, 18 hours, one day, two days, three days, one week, two weeks, three weeks, one month, two months, three months, or even six months or more. This process is expected to decrease the level of infectious viruses within the ATM. Water-replaced ATM can also, or alternatively, be exposed to γ-, x-, e-beam, and/or ultra-violet (wavelength of 10 nm to 320 nm, e.g., 50 nm to 320 nm, 100 nm to 320 nm, 150 nm to 320 nm, 180 nm to 320 nm, or 200 nm to 300 nm) radiation in order to decrease the level of, or eliminate, viable bacteria and/or fungi and/or infectious viruses. More important than the dose of radiation that an ATM is exposed to is the dose absorbed by the ATM. While for thicker ATM, the dose absorbed and the exposure dose will generally be close, in thinner ATM the dose of exposure may be higher than the dose absorbed. In addition, if a particular dose of radiation is administered at a low dose rate over a long period of time (e.g., two to 12 hours), more radiation is absorbed than if it is administered at a high dose rate over a short period of time (e.g., 2 seconds to 30 minutes). One of skill in the art will know how to test for whether, for a particular ATM, the dose absorbed is significantly less than the dose to which the ATM is exposed and how to account for such a discrepancy in selecting an exposure dose. Appropriate absorbed doses of γ-, x-, or e-beam irradiation can be 6 kGy-40 kGy, e.g., 8 kGy-38 kGy, 10 kGy-36 kGy, 12 kGy-34 kGy. Thus, the dose of γ-, x-, and or e-beam irradiation can be, for example, 12, 13, 14, 15, 16, 17, 18, 19, 20, 21, 22, 23, 24, 25, 26, 27, 28, 29, 30, 31, 32, 33, or 34 kGy. In addition, the irradiation of the water-replaced ATM can be the second or even third exposure of the ATM to irradiation. Thus, the tissue from which the ATM is made can have been irradiated (at any of the above doses) (a) prior to any of the processing steps or (b) at any stage of the processing.

Where a water-replaced ATM is subjected to both elevated temperature and irradiation, the two treatments can be performed simultaneously or sequentially, either being first. Where the treatments are performed sequentially, the second can be performed immediately after the first or there can be time gap between the treatments. This time gap can be short (e.g., about one to about 60 minutes or about one to about 11 hours) or long (e.g., about 12 to about 23 hours , about one to about six days, about 1 week to about four weeks, or about one month to about six months).

As used herein, a process (see above) used to inactivate or kill “substantially all” microorganisms (e.g., bacteria, fungi (including yeasts), and/or viruses) in ATM, particularly water-replaced ATM, is a process that reduces the level in the ATM of microorganisms by least 10-fold (e.g., at least: 100-fold; 1,000-fold; 10⁴-fold; 10⁵-fold; 10⁶-fold; 10⁷-fold; 10⁸-fold; 10⁹-fold; or even 101⁰-fold) compared to the level in the ATM prior to the process.

Generally, the water-replaced ATM are rehydrated prior to grafting or implantation. Alternatively, they can be grafted or implanted without prior rehydration; in this case rehydration occurs in vivo. Rehydration is performed by, first optionally rinsing off excess WRR solution, and then immersing the water-replaced ATM in any of the rehydration solutions described above that are used for rehydrating freeze-dried ATM. The water-replaced ATM is incubated in the solution for sufficient time for the ATM to become fully hydrated or to regain substantially the same amount of water as the tissue from which the ATM was made contains. Also, if the water replacement process resulted in shrinkage of the ATM, the water-replaced ATM is incubated in the rehydration solution for sufficient time for the ATM to revert to substantially the same volume it had prior to the water replacement process. Generally, the incubation time in the rehydration solution will be from about two minutes to about one hour, e.g., about five minutes to about 45 minutes, or about 10 minutes to about 30 minutes. The rehydration solution can optionally be replaced with fresh solution as many times as desired. This can be desirable where one or more of the water-replacing agents used in the water replacement process is not biologically compatible or is toxic. The temperature of the incubations will generally be ambient (e.g., room) temperature or can be at from about 15° C. to about 40° C., e.g., at about 20° C. to about 35° C., and the vessel containing the ATM and rehydration solution can be agitated gently during the incubation if so desired.

Generally, the water-replaced ATM is transported to the appropriate hospital or treatment facility prior to rehydration and the rehydration is performed by clinical personnel immediately prior to grafting or implanting. However, rehydration can be performed prior to transportation to the hospital or treatment facility; in this case the ATM will generally be transported under refrigerated conditions. Transportation may be accomplished via standard carriers and under standard conditions relative to normal temperature exposure and delivery times.

Methods of Treatment

The form of ATM used in any particular instance will depend on the tissue or organ to which it is to be applied.

Sheets of ATM (optionally cut to an appropriate size) can be, for example: (a) wrapped around a tissue or organ that is damaged or that contains a defect; (b) placed on the surface of a tissue or organ that is damaged or has a defect; or (c) rolled up and inserted into a cavity, gap, or space in the tissue or organ. Such cavities, gaps, or spaces can be, for example: (i) of traumatic origin, (ii) due to removal of diseased tissue (e.g., infarcted myocardial tissue), or (iii) due to removal of malignant or non-malignant tumors. The ATM can be used to augment or ameliorate underdeveloped tissues or organs or to augment or reconfigure deformed tissues or organs. One or more (e.g., one, two, three, four, five, six, seven, eight, nine, ten, 12, 14, 16, 18, 20, 25, 30, or more) such strips can be used at any particular site. The grafts can be held in place by, for example, sutures, staples, tacks, or tissue glues or sealants known in the art. Alternatively, if, for example, packed sufficiently tightly into a defect or cavity, they may need no securing device. Particulate ATM can be suspended in a sterile pharmaceutically acceptable carrier (e.g., normal saline) and injected via hypodermic needle into a site of interest. Alternatively, the dry powdered matrix or a suspension can be sprayed onto into or onto a site or interest. A suspension can be also be poured into or onto particular site. In addition, by mixing the particulate ATM with a relatively small amount of liquid carrier, a “putty” can be made. Such a putty, or even dry particulate ATM, can be layered, packed, or encased in any of the gaps, cavities, or spaces in organs or tissues mentioned above. Moreover, a non-particulate ATM can be used in combination with particulate ATM. For example, a cavity in bone could be packed with a putty (as described above) and covered with a sheet of ATM.

It is understood that an ATM can be applied to a tissue or organ in order to repair or regenerate that tissue or organ and/or a neighboring tissue or organ. Thus, for example, a strip of ATM can be wrapped around a critical gap defect of a long bone to generate a perisoteum equivalent surrounding the gap defect and the periosteum equivalent can in turn stimulate the production of bone within the gap in the bone. Similarly, by implanting an ATM in an dental extraction socket, injured gum tissue can be repaired and/or replaced and the “new” gum tissue can assist in the repair and/or regeneration of any bone in the base of the socket that may have been lost as a result, for example, of tooth extraction. In regard to gum tissue (gingiva), receding gums can also be replaced by injection of a suspension, or by packing of a putty of particulate ATM into the appropriate gum tissue. Again, in addition to repairing the gingival tissue, this treatment can result in regeneration of bone lost as a result of periodontal disease and/or tooth extraction. Compositions used to treat any of the above gingival defects can contain one or more other components listed herein, e.g., demineralized bone powder, growth factors, or stem cells.

Both non-particulate and particulate ATM can be used in combination with other scaffold or physical support components. For example, one or more sheets of ATM can be layered with one or more sheets made from a biological material other than ATM, e.g., irradiated cartilage supplied by a tissue bank such as LifeNet, Virginia Beach, Va., or bone wedges and shapes supplied by, for example, the Osteotech Corporation, Edentown, N.J. Alternatively, such non-ATM sheets can be made from synthetic materials, e.g., polyglycolic acid or hydrogels such as that supplied by Biocure, Inc., Atlanta, Ga. Other suitable scaffold or physical support materials are disclosed in U.S. Pat. No. 5,885,829. It is understood that such additional scaffold or physical support components can be in any convenient size or shape, e.g., sheets, cubes, rectangles, discs, spheres, or particles (as described above for particulate ATM).

Active substances that can be mixed with particulate ATM or impregnated into non-particulate ATM include bone powder, demineralized bone powder, and any of those disclosed above.

Factors that can be incorporated into the matrices, administered to the placement site of an ATM graft, or administered systemically include any of a wide range of cell growth factors, angiogenic factors, differentiation factors, cytokines, hormones, and chemokines known in the art. Any combination of two or more of the factors can be administered to a subject by any of the means recited below. Examples of relevant factors include fibroblast growth factors (FGF) (e.g., FGF1-10), epidermal growth factor, keratinocyte growth factor, vascular endothelial cell growth factors (VEGF) (e.g., VEGF A, B, C, D, and E), platelet-derived growth factor (PDGF), interferons (IFN) (e.g., IFN-α, β, or γ), transforming growth factors (TGF) (e.g., TGFα or β), tumor necrosis factor-α, an interleukin (IL) (e.g., IL-1-IL-18), Osterix, Hedgehogs (e.g., sonic or desert), SOX9, bone morphogenic proteins, parathyroid hormone, calcitonin prostaglandins, or ascorbic acid.

Factors that are proteins can also be delivered to a recipient subject by administering to the subject: (a) expression vectors (e.g., plasmids or viral vectors) containing nucleic acid sequences encoding any one or more of the above factors that are proteins; or (b) cells that have been transfected or transduced (stably or transiently) with such expression vectors. In the expression vectors coding sequences are operably linked to one or more transcription regulatory elements (TRE). Cells used for transfection or transducion are preferably derived from, or histocompatible with, the recipient. However, it is possible that only short exposure to the factor is required and thus histo-incompatible cells can also be used. The cells can be incorporated into the ATM (particulate or non-particulate) prior to the matrices being placed in the subject. Alternatively, they can be injected into an ATM already in place in a subject, into a region close to an ATM already in place in a subject, or systemically.

Naturally, administration of the ATM and/or any of the other substances or factors mentioned above can be single, or multiple (e.g., two, three, four, five, six, seven, eight, nine, 10, 15, 20, 25, 30,.35, 40, 50, 60, 80, 90, 100, or as many as needed). Where multiple, the administrations can be at time intervals readily determinable by one skilled in art. Doses of the various substances and factors will vary greatly according to the species, age, weight, size, and sex of the subject and are also readily determinable by a skilled artisan.

Conditions for which the matrices can be used are multiple. Thus, for example, they can be used for the repair of bones and/or cartilage with any of the above-described damage or defects. Both particulate and non-particulate ATM can be used in any of the forms and by any of the processes listed above. Bones to which such methods of treatment can be applied include, without limitation, long bones (e.g., tibia, femur, humerus, radius, ulna, or fibula), bones of the hand and foot (e.g., calcaneas bone or scaphoid bone), bones of the head and neck (e.g., temporal bone, parietal bone, frontal bone, maxilla, mandible), or vertebrae. As mentioned above, critical gap defects of bone can be treated with ATM. In such critical gap defects, the gaps can be filled with, example, a putty of particulate ATM or packed sheets of ATM and wrapped with sheets of ATM. Alternatively, the gaps can be wrapped with a sheet of ATM and filled with other materials (see below). In all these bone and/or cartilage treatments, additional materials can be used to further assist in the repair process. For example, the gap can be filled cancellous bone and or calcium sulfate pellets and particulate ATM can be delivered to sites of bone damage or bone defects mixed with demineralized bone powder. In addition, ATM can be combined with bone marrow and/or bone chips from the recipient.

ATM can also be used to repair fascia, e.g., abdominal wall fascia or pelvic floor fascia. In such methods, strips of ATM are generally attached to the abdominal or pelvic floor by, for example, suturing either to the surrounding fascia or host tissue or to stable ligaments or tendons such as Cooper's ligament.

Infarcted myocardium is another candidate for remodeling repair by ATM. Contrary to prior dogma, it is now known that not all cardiac myocytes have lost proliferative and thus regenerative potential [e.g., Beltrami et al. (2001) New. Engl. J. Med. 344:1750-1757; Kajstura et al. (1998) Proc. Nat'l. Acad. Sci. USA 95:8801-8805]. Moreover, stem cells, present for example in bone marrow and blood and as pericytes associated with blood vessels, can differentiate to cardiac myocytes. Either the infarcted tissue itself can be removed and replaced with a sheet of ATM cut to an appropriate size or a suspension of particulate ATM can be injected into the infarcted tissue. Congenital heart hypoplasia, or other structural defects, can be repaired by, for example, making an incision in the tissue, expanding the gap created by the incision, and inserting a sheet of ATM cut to the desired size, or placing sheets of ATM on the epicardial and endocardial surfaces and placing particulate ATM between them. It is understood that, in certain conditions, creating a gap by incision may not be sufficient and it may be necessary to excise some tissue. Naturally, one of skill in the art will appreciate that the ATM can be used similarly to repair damage to, or defects in, other types of muscle, e.g., ureter or bladder or skeletal muscle such as biceps, pectoralis, or latissimus.

Moreover, sheets of ATM can be used to repair or replace damaged or removed intestinal tissue, including the esophagus, stomach, and small and large intestines. In this case, the sheets of ATM can be used to repair perforations or holes in the intestine. Alternatively, a sheet of ATM can be formed, for example, into a cylinder which can be used to fill a gap in the intestine (e.g., a gap created by surgery to remove a tumor or a diseased segment of intestine). Such methods can be used to treat, for example, diaphragmatic hernias. It will be understood that an ATM in sheet form can also be used to repair the diaphragm itself in this condition as well as in other conditions of the diaphragm requiring repair or replacement, or addition of tissue.

The following examples serve to illustrate, not limit, the invention.

### EXAMPLES

### Example 1

### Acellular Dermal Matrices (ADM)

In the experiments described in Examples 2-6 below, ADM were produced using LifeCell's proprietary methodology. The methodology for making ADM is broadly described in this example and details for the ADM used in individual experiments are provided in the relevant examples. The description below was that used for the production of ADM from human skin. Except where otherwise stated, an essentially identical process was used for the production of ADM from pig skin.

Human donor skin was obtained from various U.S. tissue banks and hospitals throughout the U.S. that collected skin samples from deceased donors after obtaining consent from family members. Procured skin was placed in RPMI 1640 tissue culture medium containing antibiotics (penicillin and streptomycin) and was shipped to LifeCell's facility in Branchburg, N.J., on wet ice, in the same medium. On arrival, the temperature of the skin tissue container wass measured and the skin tissue was discarded if the temperature was above 10° C. The RPMI 1640 medium was changed under aseptic condition and the skin was stored at 4° C. while serological tests for various pathogens (Treponema pallidum (tested for by the RPR and VDRL methods), HIV (human immunodeficiency virus) I and II, hepatitis B virus, hepatitis C virus, and HTLV (human T-lymphotropic virus) I and II) were performed on a sample of the skin. The skin was discarded if any of the pathogens were detected. Otherwise, it was transferred to a pre-freezing aqueous solution of 35% weight to volume (w/v) maltodextrin (M180) in phosphate buffered saline (PBS). After 2 to 4 hours at room temperature (20 to 25° C.), the solution containing the skin was frozen at −80° C. and stored in a −80° C. freezer until it was processed as described below.

Frozen skin with pre-freezing solution was thawed at 37° C. in a water bath until no ice was visible. The pre-freezing solution was drained and the skin was submitted to the following processing steps: (i) de-epidermization; (ii) de-cellularization; (iii) wash.

(i) De-epidermization: Skin epidermis was removed by incubating the tissue sample with gentle agitation in a de-epidermizing solution (1M NaCl, 0.5% w/v Triton X100, 10 mM ethylenediaminetetraacetic acid (EDTA)) for 8-32 hours at room temperature. For processing of pig skin, this incubation was performed for 30-60 hour at room temperature. The epidermal layer was physically removed from dermis. The epidermis was discarded and the dermis was subjected to further processing.

(ii) Decellularization: In order to kill in cells and remove cellular components and debris, the dermis was rinsed for 5 to 60 minutes with a decellularizing solution (2% w/v sodium deoxycholate, 10 mM EDTA, 10 mM HEPES buffer, pH 7.8-8.2) and then incubated with gentle agitation in a fresh lot of the same solution for 12-30 hours at room temperature.

(iii) Wash: The washing regimen serves to wash out dead cells, cell debris, and residual chemicals used in the previous processing steps. The decellularized dermis was transferred to a first wash solution (phosphate buffered saline (PBS) containing 0.5% w/v Triton X-100 and 10 mM EDTA) which was then incubated with gentle agitation for 5 to 60 minutes at room temperature. The dermis was then subjected to three sequential washes in a second wash solution (PBS containinglo mM EDTA) with gentle agitation at room temperature. The first two washes were short (15-60 minutes each) and the third wash was long (6-30 hours).

After the wash regimen, the resulting ADM were cut into appropriate sizes and then used for the experiments described in Examples 2-6.

### Example 2

### Water Replacement In ADM By Glycerol

Incubation times for the three processing steps (see Example 1) performed in making the ADM used in the experiments described in this example were as follows: (i) 19 hours; (ii) 13 hours; and (iii) (a) 15 minutes in the first wash solution, (b) 15 minutes in the second wash solution; (c) 15 minutes in the second wash solution; and (d) 15 hours in the second wash solution.

Three ADM samples (after step (iii) of the above-described processing procedure) were separately incubated in normal saline (0.9% w/v NaCl in water) solutions of 20% volume to volume (v/v) glycerol, of 30% v/v glycerol, or of 40% v/v glycerol for 80 minutes at room temperature. The ADM samples shrunk slightly in the glycerol solutions but no difference in shrinkage was observed between the samples. Then, each of the three ADM samples was transferred to a separate 60% v/v glycerol in normal saline solution. The ADM samples that were initially treated in the 20% glycerol solution shrunk the most in the 60% v/v glycerol solution. After the treatment in 60% glycerol solution, each of the three ADM samples was further treated in a separate 85% v/v glycerol in normal saline solution. The final sizes (area) of samples were 75%, 72% and 84% of those measured prior to the initial glycerol treatment for the ADM samples initially treated with 20%, 30%, and 40% glycerol, respectively. Thus, ADM samples that were initially exposed to 40% v/v glycerol showed the least shrinkage after subsequent treatments at higher concentrations of glycerol.

Two ADM samples, each with a different thickness and derived from a different human donor, were used to investigate the kinetics of water replacement. Glycerol content within the ADM was measured using the refractive index method. The “refractive index” of a solution is related to its concentration. The Palette Series PR-201 Digital Refractometer (Atago U.S.A., Inc., Kirkland, Wash.) is designed to measure the concentration of a solute or a solvent in a liquid solution. It can measure the range from Brix 0.0% to 60% with an accuracy of +0.2% and has automatic temperature compensation between 10° C. and 40° C. The refractometer displays glycerol concentration on the Brix (%) scale. Standard curves were established for glycerol/saline solutions. To measure glycerol content in the tissue matrix the sample is incubated in a known volume of normal saline solution. After equilibration, the glycerol concentration in the incubation solution is measured. From this value, the amount of glycerol in the sample can be determined.

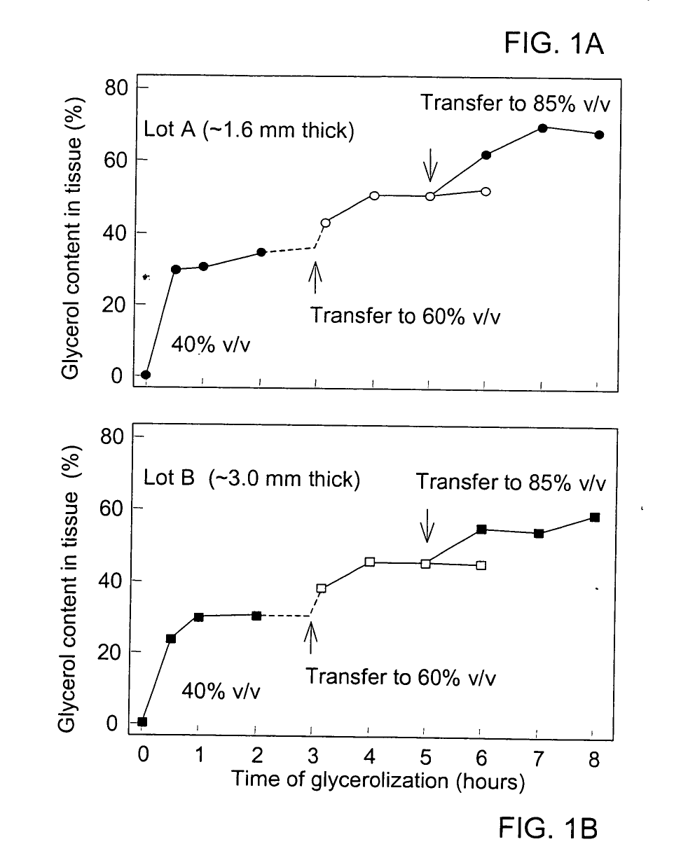

The average thickness of the two ADM samples tested was approximately 1.6 mm and 3.0 mm, respectively. Both the ADM samples were incubated sequentially in separate normal saline solutions of 40% v/v, 60% v/v, and 85% v/v glycerol for different periods of time. One hour was sufficient to achieve equilibrium in 40% v/v and 60% v/v glycerol solutions (FIG. 1). Two to three hours was required to reach equilibrium in the 85% glycerol solutions. The final ADM products consisted of, on a weight to weight (w/w) basis, about 8% water, about 20% to 30% tissue matrix, and about 60% to 70% glycerol. Glycerol content in the tissue matrix was affected by the density and initial hydration of ADM. In this experiment, the thicker (about 3 mm thick) ADM had a lower final glycerol concentration (about 60% w/w) than the thinner (about 1.6 mm thick) ADM (about 70% w/w).

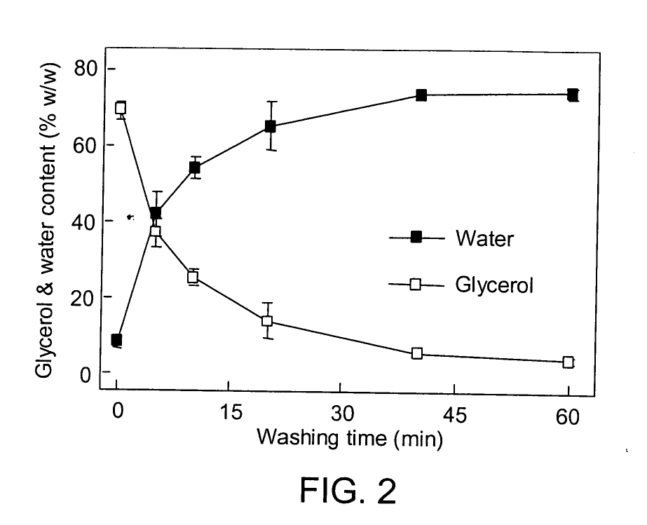

Water replacement in ADM samples made using all the above-described methods was fully reversible. Glycerol in the ADM products after the incubation in the highest concentration of glycerol (85%) was rapidly replaced by water upon rehydration in normal saline (0.9% w/v NaCI) (see, e.g., FIG. 2). Since glycerol solutions have a refractive index close to that of skin tissue (˜1.34 to 1.44), the glycerolized ADM are transparent. When rehydrated, the transparent glycerolized ADM reverted to their original opaque appearance and to their original dimensions, i.e., shrinkage in the ADM that was observed in any of the above-described methods was fully reversible.

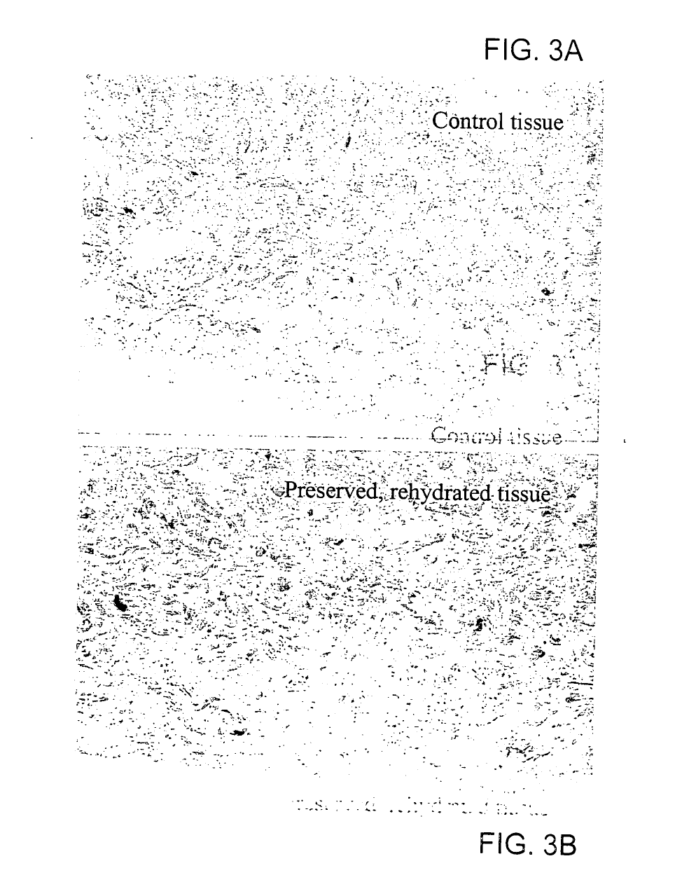

Glycerolized ADM samples were rehydrated in normal saline and then fixed with 10% formalin for structural examination using hemotoxylin and eosin (H & E) staining. No structural alteration was observed after water replacement and rehydration treatment (FIG. 3). ADM histology was well preserved. Some ADM samples were glycerolized and rehydrated two times to amplify possible structural alterations by the above-described glycerolization and rehydration method. Again the rehydrated ADM samples showed the typical mesh network without separation or condensation of the tissue matrix and the tissue matrix structures were the same as the samples that had not been subjected to water replacement and rehydration.

### Example 3

### γ-Irradiation of the Preserved ADM

It is known that γ-irradiation damages collagen-based tissue matrices. One of the damaging mechanisms involves homolytic water splitting with hydroxyl radical formation and heterolytic transfer of electrons to oxygen that causes reactive oxygen radical formation. Tissue damage is due to free radical-mediated oxidative events. Previous studies showed that 12 kGy γ-irradiation, applied either after freeze-drying or before freeze-drying, consistently lead to the failure of ADM (prepared as described in Example 1 and subsequently freeze-dried) to pass a Quality Control (QC) test developed at LifeCell, Inc. This QC test is described in Example 8 below. In addition, unrelated studies suggested that glycerol might stabilize tissues against radiation damage.

Incubation times for the three processing steps (see Example 1) performed in making the ADM used in the experiment described in this example were as follows: (i) 12 hours; (ii) 15 hours; (iii) (a) 30 minutes in the first wash solution, (b) 15 minutes in the second wash solution, (c) 15 minutes in the second wash solution, and (d) 23 hours in the second wash solution.

ADM samples were incubated sequentially in separate normal saline solutions of 40% v/v glycerol for 2 hours, of 60% v/v glycerol for 2 hours, and of 85% v/v glycerol for 3 hours. Water content of the ADM was reduced from 85%-90% w/w to about 8% w/w. The glycerolized samples were γ-irradiated at ˜80° C. with dosages of 0, 12, 18, or 24 kGy. After irradiation, the samples were rehydrated in normal saline and fixed with 10% formalin for structural examination using H & E staining.

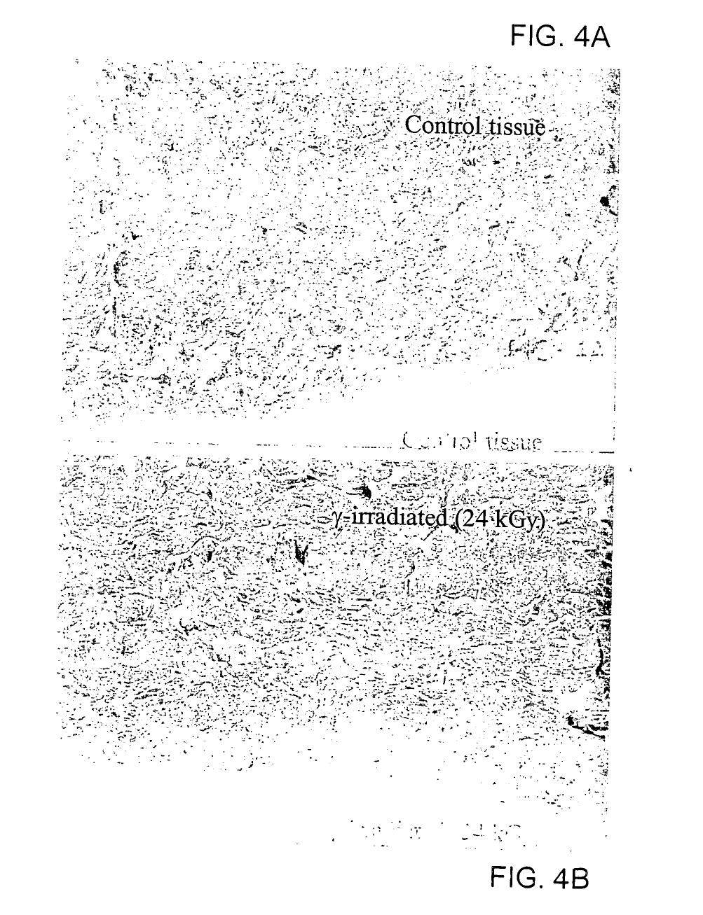

This experiment showed that water replacement increased the resistance of ADM to γ-irradiation. At 12 kGy, there was only minor structural alteration in papillary and reticular layers of the ADM (e.g., a slight increase in collagen bundle separation). Even after γ-irradiation with 18 kGy and 24 kGy (FIG. 4), the relevant water-replaced and rehydrated ADM demonstrated good structural preservation.

### Example 4

### Implantation of the Preserved ADM Into Nude Mice

Incubation times for the three processing steps (see Example 1) used for making the ADM used in the experiment described in this example were as follows: (i) 16 hours; (ii) 12 hours; (iii) (a) 18 minutes in the first wash solution, (b) 17 minutes in the second wash solution, (c) 18 minutes in the second wash solution, and (d) 10 hours in the second wash solution.

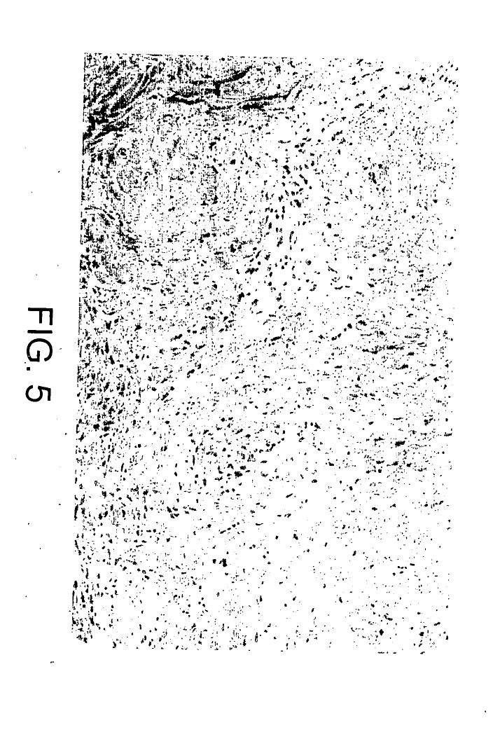

After the step (iii) of the above-described processing procedure, the ADM was cut into samples of about 1.0 square centimeter. The samples were incubated in normal saline solutions containing 40% v/v glycerol for 3.5 hours, 70% v/v glycerol for 2 hours, and 85% v/v glycerol for 2.5 hours. The samples were stored in sterile freezing vials for 4 days at room temperature. The vials were wrapped with aluminum foil to prevent exposure to light during storage. The samples were rehydrated in normal saline for 30 to 40 minutes and then implanted subcutaneously into nude mice. Mice were sacrificed after 21 days and the implants were removed and fixed in 10% formalin for histological examination using H & E staining. The ADM implants showed rapid host cell repopulation and re-vascularization (FIG. 5).

### Example 5

### Thermal Treatment of the Preserved ADM

Incubation times for the three processing steps (see Example 1) used for making the ADM used in the experiment described in first part of this example were as follows: (i) 26 hours; (ii) 20 hours; (iii) (a) 60 minutes in the first wash solution, (b) 30 minutes in the second wash solution, (c) 30 minutes in the second wash solution, and (d) 18 hours in the second wash solution.

After the step (iii) of the above-described processing procedure, the ADM was cut into samples of about 1.0 square centimeter. The samples were treated sequentially in normal saline solutions containing 40% v/v glycerol for 2 hours, 55% v/v glycerol for 1.5 hours, 70% v/v glycerol for 1.5 hours, and 85% v/v glycerol for more than 72 hours. At the end of each glycerolization step, an ADM sample was kept and stored at 4° C. for later testing. Thermal stability of the various glycerol-treated ADM samples was determined using differential scanning calorimetry (DSC). The ADM samples (each about 20 mg) were hermetically sealed in DSC crucibles, and heated at a scanning rate of 1° C./min. DSC measures the heat flow in a sample. The melting (denaturation) of collagen and other proteins is an endothermic transition event and therefore absorb energy during the melting transition. A DSC thermogram is a plot of heat flow against temperature, from which the onset transition temperature (Tm) and the enthalpy (ΔH) of melting are determined. The onset Tm is an indicator of thermal stability of proteins in the processed ADM.

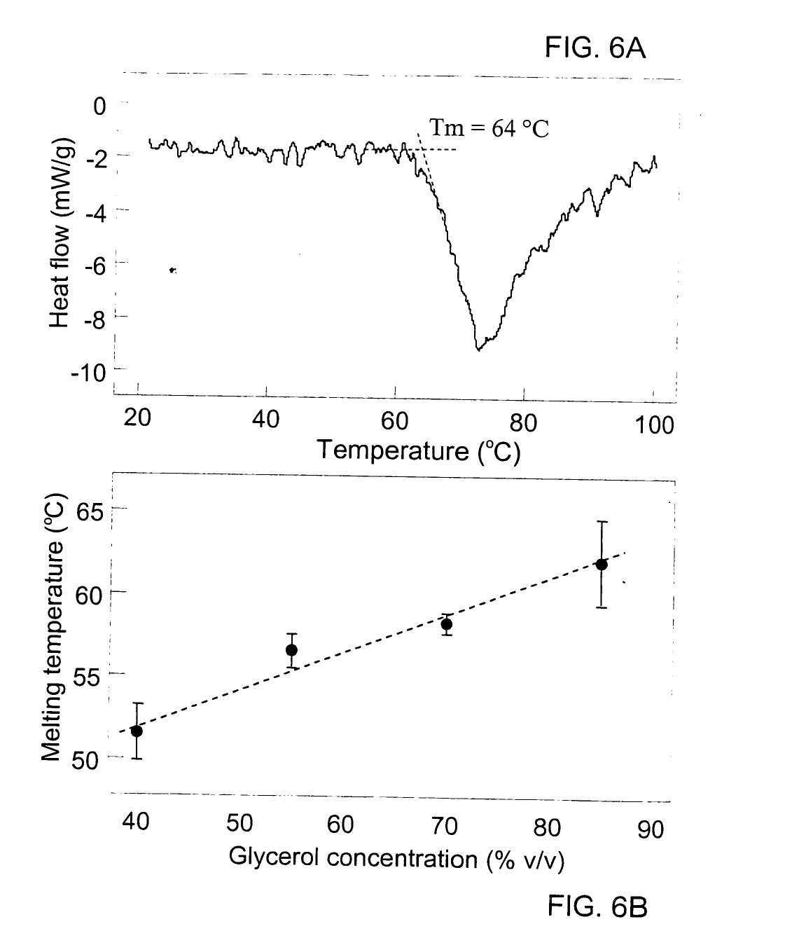

The Tm of the fully hydrated ADM was typically found to be 40° C. to 45° C. FIG. 6A shows a DSC thermogram of an ADM sample in which about 92% of the water in the sample was replaced with glycerol. The water replacement process increased the Tm to about 4° C. The increase in thermal stability of processed ADM is proportionally related to the extent of water replacement. Increasing the amount of water replaced by glycerol resulted in increases in Tm (FIG. 6B). The onset Tm of ADM was found to increase to 60° C.-65° C. after 90% water replacement.

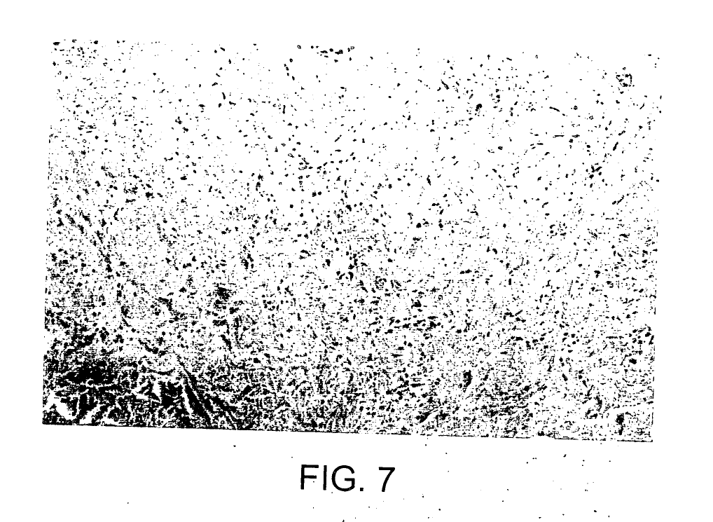

In vivo performance of preserved and heated ADM was evaluated using nude mice. Incubation times for the three processing steps (see Example 1) used for making the ADM used in the in vivo experiment described in this example were as follows: (i) 16 hours; (ii) 12 hours; (iii) (a) 18 minutes in the first wash solution, (b) 17 minutes in the second wash solution, (c) 18 minutes in the second wash solution, and (d) 10 hours in the second wash solution. After the step (iii) of the processing procedure, the ADM was cut into samples of about 1.0 square centimeter. The samples were sequentially treated in normal saline solutions containing 40% v/v glycerol for 3.5 hours, 70% v/v glycerol for 2 hours, and 85% v/v glycerol for 2.5 hours. Treated samples were stored in sterile vials, which were wrapped in aluminum foil to prevent exposure to light, and stored at an elevated temperature (an average temperature of 55° C., fluctuating between 52° C. and 59° C.) for 4 days. After rehydration in normal saline for 30 to 40 minutes, the samples were implanted subcutaneously into nude mice. Mice were sacrificed after 21 days and the implants were removed and fixed in 10% formalin for histological examination using H & E staining. Host cell repopulation and vascularization of the explanted ADM were evaluated. Water replacement with glycerol increased the resistance of the ADM to thermal damage. Even after being stored at an elevated temperature (52° C. to 59° C.) for 4 days, the glycerolized and rehydrated ADM showed significant host cell infiltration and re-vascularization (FIG. 7). When “control damaged” ADM were implanted no detectable cell infiltration, re-vascularization, or remodelling occurred. The “control damaged” ADM included those had not undergone water replacement and: (a) had been treated with guanidine hydrochloride; or (b) had been stored at room temperature and exposed to light for at least four years.

### Example 6

### Water Replacement In ADM Using Other Hydrophilic Compounds

Incubation times used for making the ADM used in the experiment described in this example were as follows: (i) 24 hours; (ii) 15 hours; (iii) 20 minutes for incubation using the first wash solution, 15 minutes for the first wash using the second wash solution, 15 minutes for the second wash using the second wash solution, 30 hours for the thrid wash using the second wash solution.

After the step (iii) of the above-described processing procedure, water in the ADM was replaced by a cocktail of liquid hydrophilic compounds. The cocktail contained 25% v/v polyethylene glycol (molecular weight, 400 daltons), 25% v/v ethylene glycol and 50% v/v glycerol. The ADM samples were sequentially treated in normal saline solutions containing 40% v/v cocktail for 1.5 hours, 55% v/v cocktail for 1.5 hours, 70% v/v cocktail for 1.5 hours, and 85% v/v cocktail for more than 72 hours. After water replacement using the cocktail, the ADM samples shrunk by about 15% to about 20%.

The glycerolized ADM were stored in 100 ml plastic bottles for 20 days at room temperature (about 22° C.). The bottles were wrapped with aluminum foil to prevent exposure to light during storage. The glycerolized ADM were rehydrated in normal saline overnight. Upon rehydration, the ADM reverted to their original volume. Rehydrated samples were fixed in 10% formalin for histological examination using H & E staining. No structural alteration in the ADM was observed after water replacement with the cocktail solution, storage, and rehydration. The rehydrated ADM demonstrated structural integrity and mechanical property similar to that of samples that had not been subjected to water replacement, storage, and rehydration.

### Example 7

### Water Replacement In Acellular Vein Matrix (AVM)

Human umbilical cords were collected and provided by the National Disease Research Interexchange (NDRI) (Philadelphia, Pa.). Tissue banks have established procurement guidelines, which are published by the American Association of Tissue Banks. These guidelines include instructions for donor selection, completion of consent forms and a caution to avoid mechanical distention or other mechanical damage to the vein during the dissection process. After harvesting, the umbilical cords were flushed with a solution consisting of 1000 ml Plasmalyte™ physiological solution supplemented with 5000 units of Heparin and 120 mg of Papaverine (1 liter per vein). The umbilical cords were placed in cold RPMI 1640 tissue culture medium (4° C.) containing antibiotics (penicillin and streptomycin) and were shipped by overnight delivery to LifeCell's facility in Branchburg, N.J., on wet ice, in the same tissue culture medium. Upon receipt of the shipped material, the container temperature was verified to be not more than 10° C. The tissue were inspected for tears, ruptures, smudges and other physical defects and submitted to the same serological tests for pathogens performed on skin samples (see Example 1). Umbilical cords that were free of physical damage, defects, and pathogens were used for further experimentation. Accepted umbilical cords were placed in vessels containing 500 mL cryopreservation solution and incubated for 16 to 32 hours at 4° C. The cryopreservation solution was 50% w/v polyalditol (PD30) in 30 mM HEPES buffer (pH 6.8 to 7.2) containing 8 mM EDTA. Other cryoproservation solutions were: (1) 35% maltodextrin (Ml 80) in 20 mM PBS (pH 6.8 to 7.2); and (2) 0.5M dimethylsulfoxide (DMSO), 0.5M propylene glycol, 0.25M 2-3 butanediol, 12% w/v sucrose, 15% w/v polyvinylpyrrolidone (PVP) and 15% w/v dextran in 20 mM PBS (pH 6.8 to 7.2). After the incubation at 4° C., the umbilical cord/cryopreservation solution mixtures were cooled to a temperature of −80° C. and stored in a −80° freezer for storage until further processing as described below.

The umbilical cords frozen in cryopreservation solution were thawed at 37° C. in a water bath until no visible ice remained. The cryoproservation solution was drained and the umbilical veins were carefully separated from the other cord tissues using surgical scissors. In order to kill cells in the veins and remove all cellular components and cell debris, dissected vein tissues were placed in a decellularization solution containing 25 mM EDTA, 1M NaCl and either 8 mM CHAPS, 1.8 mM sodium dodecylsulfate (SDS) (or 2% w/v n-Octyl glucopyranoside) in sterile PBS and incubated with gentle agitation in the same solution for 20 hours at room temperature. The decellularized vein tissues were washed with PBS containing 10 mM EDTA with gentle agitation at room temperature three times (30 minutes each wash) resulting in acellular vein matrices (AVM).

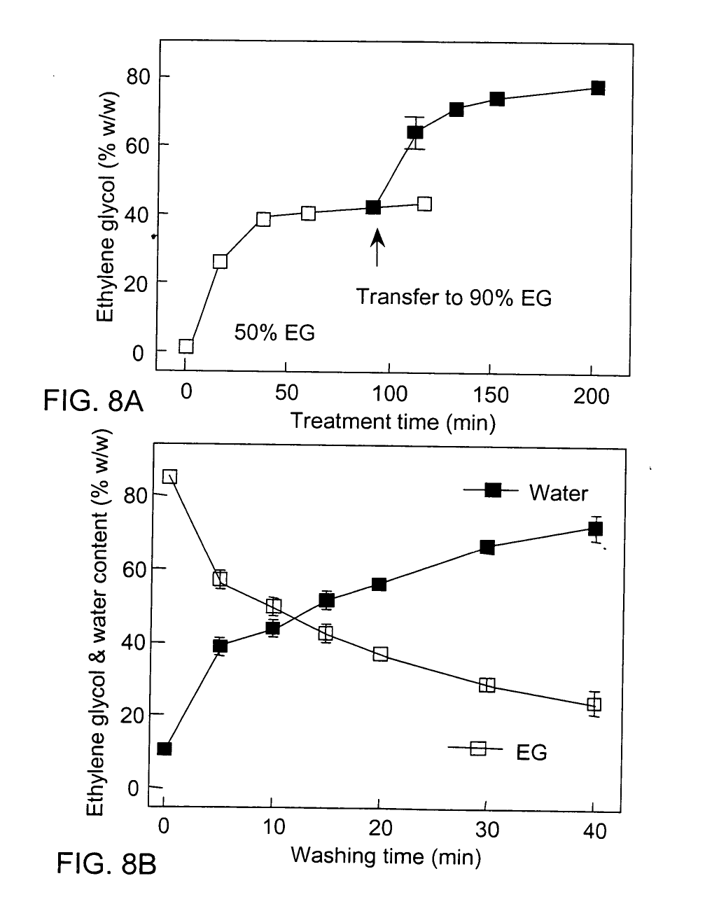

Water replacement method #1: This experiment consisted of the following two sequential water replacement steps: (1) an AVM produced as described above was incubated with gentle agitation in 50% v/v ethylene glycol saline solution at room temperature for 1 hour; (2) the AVM was transferred to a solution of 90% v/v ethylene glycol saline solution and incubated at room temperature for 2 hours. Three replicate AVM samples were taken at each of various time points during both steps of the process and the EG concentrations in all the samples were measured using the refractive index method (described above). FIG. 8A shows the influx of EG into the AVM. AVM treated with ethylene glycol (EG) saline solutions readily equilibrated with the solutions. Sixty minutes was sufficient for the AVM to equilibrate with the 50% v/v EG solution and 90 to 120 minutes was sufficient for the 50% v/v EG treated AVM to equilibrate in the 90% v/v EG solution. The water replacement process reduced the water content of the AVM from about 97% w/w to about 7% w/w and resulted in an ethylene glycol content in the AVM of about 80% to about 85% w/w. Moreover, the process resulted in a decrease in volume of the AVM by 40% to 60%.

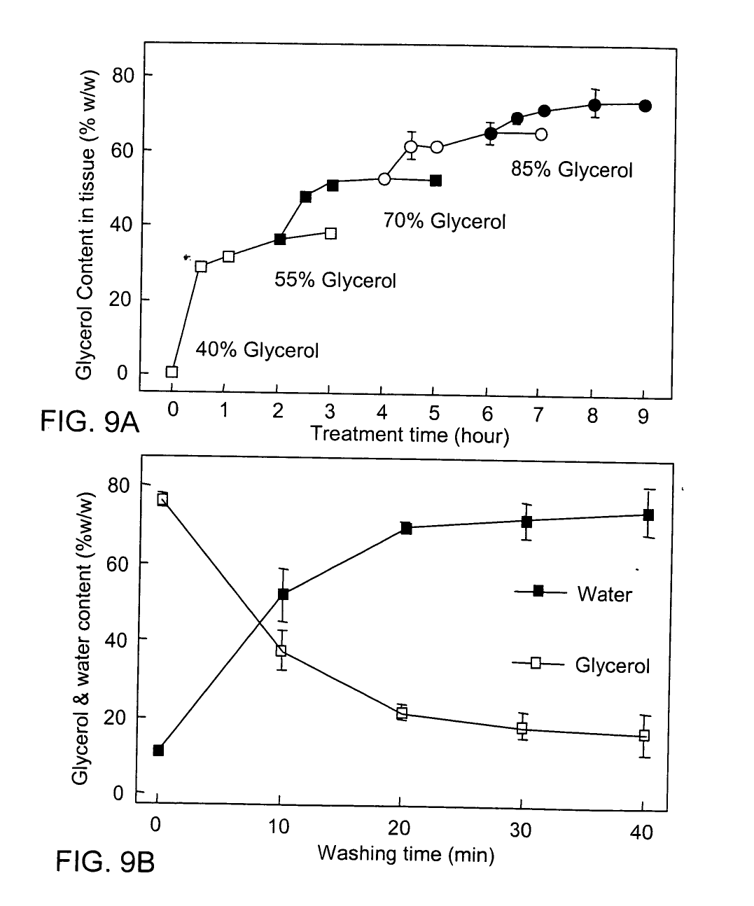

Water replacement method #2: This experiment consisted of the four water replacement steps. AVM samples being incubated sequentially in solutions of 40% v/v glycerol for 1 hour, of 55% v/v glycerol for 1 hour, of 70% v/v glycerol for 1 hour, and of 85% v/v glycerol for 2 hours at room temperature (˜22° C.). Three replicate AVM samples were taken at each of various time points during the entire water replacement process, and the glycerol concentrations in all the samples were measured using the refractive index method (described above). FIG. 9A shows the influx of glycerol into AVM during a four-step water replacement process. AVM treated with glycerol saline solutions readily equilibrated with the solutions. Sixty minutes was sufficient for the AVM to equilibrate in the 40% v/v and 55% v/v solutions, whereas 90 to 120 minutes was needed for the treated AVM to equilibrate in higher concentrations (i.e., 70% v/v and 85% v/v). After the glycerol treatments, water content in the AVM samples was reduced from 97% to 12% w/w and the glycerol content of the AVM was 75% to 80% w/w. The process decreased the AVM volume by 30%-40%.

Water replacement method #3: AVM samples were incubated with gentle agitation in a solution of 30% v/v glycerol in normal saline for 2 hours at room temperature (˜22° C.). They were then transferred to a solution of 75% v/v glycerol in normal saline and incubated for 4 hours at room temperature (˜22° C.). The treated AVM samples were placed in 25 ml glass bottles containing 15 mL of solution of 85% v/v glycerol in normal saline and stored at room temperature for 7 weeks. The bottles were wrapped with aluminum foil to exclude light. Residual water content, glycerol concentration within AVM and volume reductions were essentially the same as those described above in water replacement method #2.

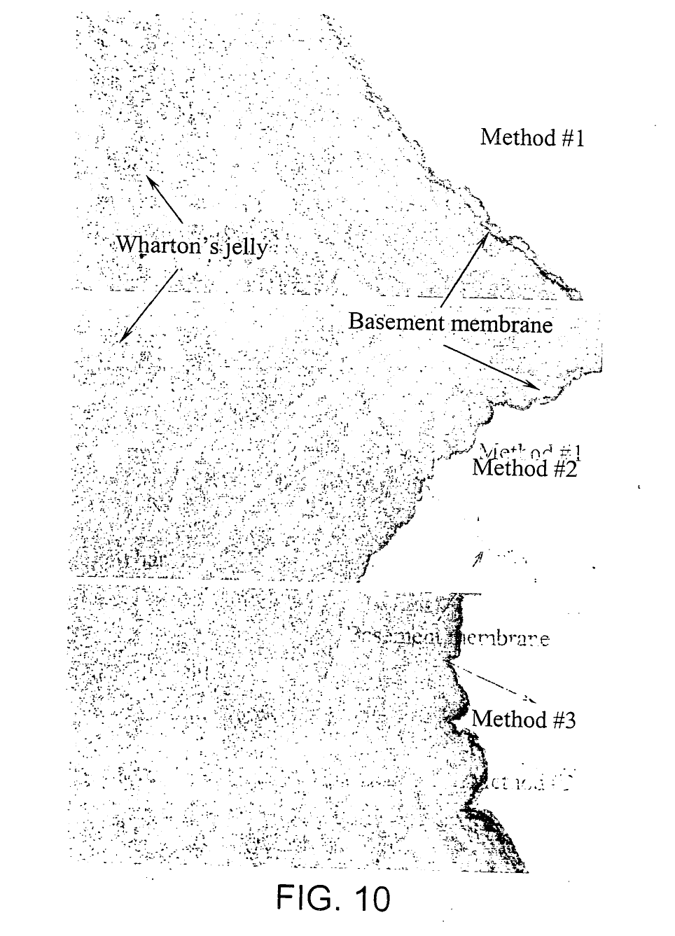

Upon rehydration in PBS or normal saline (0.9% NaCl), the amount of water-replacing agents in the AVM decreased rapidly (FIG. 8B and FIG. 9B). The shrinkage of AVM samples observed during water replacement treatment was fully reversed upon rehydration. After rehydration for 1 hour, AVM samples were fixed in 10% formalin for histological evaluation by H & E and Verhoeff's staining. Analysis of the rehydrated AVM showed that all three water replacement methods preserved the structural integrity of vein extracellular matrix (FIG. 10). The integrity of the basement membrane, lumen, and Wharton's jelly was well preserved. In circumferential, compliance and burst tests the test AVM performed comparably to control AVM that had not been subjected to water replacement, storage, and rehydration.

### Example 8

### Quality Control Analysis of ADM

The following is a summary of the Quality Control procedure used by for assessing the quality of the ADM. The methodology, or obvious variations of it, can be used for assessing the quality of ATM produced from a variety of collagen-containing tissues and to assess the effect of the water-replacing process of the invention on such ATM.

Sections of an ADM are mounted on glass microscope slides and stained with H & E using standard procedures. The following microscopic analysis is then performed on these sections.

1. The slides are examined for the presence of epidermal cell remnants. The presence of any identifiable epidermal cell remnant (above the basement membrane) is unacceptable and the relevant ADM lot is rejected.

2. The slides are examined for the presence of dermal cell (e.g., fibroblast) remnants. If any cell remnants are noted and immunostaining of separate sections for the presence of major histocompatibility complex (MHC) class I and class II antigens molecules gives negative results, two additional samples of the ADM lot should be processed for MHC class I & II as well as H & E analysis. The slides from all three samples should be reviewed. If the results from all three samples are inconclusive, samples are sent for electron microscopy analysis for final assessment of whether the ADM contains cell remnants.

3. Histological analysis of ADM samples is designed to test for the presence of an intact matrix. Samples are scored using the following criteria:

3.1. Presence of Holes in the Sample: Holes in the ADM may represent a variety of structures including blood vessels, empty adipocytes, vacant hair follicles, and expansion of gas bubbles within the sample during the freeze-drying process. Histologically, it is difficult to distinguish between these, and hence the presence of holes is graded according to the total percentage area of the sample occupied by these structures. Lots with holes encompassing more than 60% of the sample are rejected. Scoring:

| Score | Assessment |
|---|---|
| 1-2 | Holes in 0%-10% of the sample. |
| 3-4 | Holes in 11%-25% of the sample. |
| 5-6 | Holes in 26%-40% of the sample. |
| 7-9 | Holes in 41%-60% of the sample. |
| 10 | Holes in >60% of the sample. |

3.2. Collagen Damage: “Collagen damage” refers to the presence of broken collagen fibers, condensed collagen fibers, or distorted fibers. Collagen damage is reported as incidence of observation in visual fields for all samples. Lots are rejected if evidence of collagen damage is observed in all samples in all visual fields. Scoring:

| Score | Assessment |
|---|---|
| 1-2 | Damage in 0%-10% of the fields examined. |
| 3-4 | Damage in 11%-25% of the fields examined. |
| 5-6 | Damage in 26%-50% of the fields examined. |
| 7-8 | Damage in 51%-75% of the fields examined. |
| 9-10 | Damage in 76%-100% of the fields examined. |

3.3. Papillary and Reticular Layer: Normal human dermis contains a papillary layer consisting of a superficial basement membrane zone and then a layer of vascular and amorphous structure lacking clearly defined thick bundles of collagen. The collagen and elastin appearance of the papillary layer is one of fine reticulation. The reticular layer merges with the papillary layer and is composed of clearly defined collagen bundles. If collapse or melting occurs during process of the tissue to produce the ADM, there will be a condensation of the papillary layer. If skin is extensively scarred or subject to a pathological process such as scleroderma or epidermolysis, there will be a loss of the papillary layer. If samples lack a papillary layer, the relevant lot is rejected. Scoring:

| Score | Assessment |
|---|---|
| 0 | Normal bilayer, clearly defined vascular |
| | plexus, clear transition. |
| 0-2 | Poorly defined undulations of rete ridge |
| | and rete peg. |
| 0-2 | Loss of structural features in superficial |
| | papillary layer, including vascular plexus. |
| 0-2 | Loss of structural features in inner |
| | papillary layer. |
| 0-2 | Loss of transition zone between papillary |
| | and reticular layer. |
| 10 | Absence or replacement of papillary layer |
| | with amorphous condensed layer. |

3.4. Collagen Orientation: The collagen orientation within the ADM should be that of a meshwork. Linear orientation of collagen can occur due to pathology (e.g., scar) or as a normal histological feature (deep reticular dermis). Samples are rated as the percent of total structure represented by linear collagen. Collagen orientation alone is not grounds for rejection. Scoring:

| Score | Assessment |
|---|---|
| 1 | Meshwork. |
| 3 | 50% meshwork/50% linear. |
| 5 | 100% linear. |

3.5. Collagen Separation: Normal collagen in an ADM should have an internal fibrous structure, and separation between bundles should represent a gradual transition from one fiber to the next. Collagen separation is a recognized change that occurs in processing. At its extreme, the collagen fiber loses its fibrous nature and appears amorphous, the separation between fibers becomes an abrupt transition, and the fibers often appear angulated. Based on animal and clinical evaluation, no functional significance can to date be attributed to this appearance. However, although not grounds for rejection alone, this is included as part of the assessment of matrix integrity. Scoring:

| Score | Assessment |
|---|---|
| 1 | No artificial separation, fibrous structure evident. |
| 3 | Sharp separation, some fibrous definition. |
| 5 | Angular separation, amorphous collagen appearance. |

Scores for each criterion of histological analysis are added. If the sum of scores is <22, the lot passes. If the sum of scores is ≧22, the lot fails. If the lot scores 10 for holes, collagen damage, or papillary to reticular ratio, it fails. The primary reviewer may request a secondary reviewer to perform additional slide reviews on any lot. The secondary reviewer scores the slide(s) independently and the mean of the two scores will be used to determine if the lot passes or fails. In addition, if both reviewers determine the lot is unacceptable for release, this decision can be made independent of the mean score. In the event of this type of failure, a written rationale is provided that justifies the decision.
3.6. Collagen Bundles: Sections of ADM are examined for the presence of collagen bundles in the dermis. If a low density of collagen bundles is noted, a Verhoeffs stain is performed to determine its relative level of elastin. The lot is considered acceptable if the corresponding elastin density is normal or high.
3.7. Digital micrographs are taken (at a magnification of 100×) of each slide, reviewed and kept with the written records of the Quality Control analysis. The micrographs should be clear representations of the samples. If a micrograph is unclear or out of focus, it is unacceptable and an additional micrograph of the relevant slide must be taken.

A number of embodiments of the invention have been described. Nevertheless, it will be understood that various modifications may be made without departing from the spirit and scope of the invention. Accordingly, other embodiments are within the scope of the following claims.

What is claimed is:

1. A composition comprising:
an isolated acellular tissue matrix; and
within the acellular tissue matrix, a water-replacing reagent,
wherein the acellular tissue matrix contains not more than 30% of the water that the matrix contains if fully hydrated.

2. The composition of claim 1, wherein the amount of water within the matrix is sufficiently low to allow storage of the composition at ambient temperatures for an extended period of time without substantial damage to the matrix.

3. The composition of claim 1, wherein the water-replacing reagent comprises glycerol.

4. The composition of claim 3, wherein the water-replacing reagent consists of glycerol.

5. The composition of claim 1, wherein the water-replacing reagent comprises one or more water-replacing agents selected from the group consisting of dimethylsulfoxide (DMSO) and polyhydroxyl compounds.

6. The composition of claim 5, wherein the polyhydroxyl compounds are selected from the group consisting of monosaccharides, disaccharides, oligosaccharides, polysaccharides, poly-glycerol, ethylene glycol, propylene glycol, polyethylene glycol (PEG), and polyvinyl alcohols (PVA).

7. The composition of claim 5, wherein the water-replacing reagent comprises glycerol and ethylene glycol.

8. The composition of claim 7, wherein the glycerol and the ethylene glycol are present in equal concentrations by weight, by volume, or by molarity.

9. The composition of claim 1, wherein the matrix comprises dermis from which all, or substantially all, viable cells have been removed.

10. The composition of claim 1, wherein the acellular matrix comprises a tissue from which all, or substantially all, viable cells have been removed, wherein the tissue is selected from the group consisting of fascia, pericardial tissue, dura, umbilical cord tissue, placental tissue, cardiac valve tissue, ligament tissue, tendon tissue, arterial tissue, venous tissue, neural connective tissue, urinary bladder tissue, ureter tissue, and intestinal tissue.

11. The composition of claim 1, wherein the acellular tissue matrix is made from human tissue.

12. The composition of claim 1, wherein the acellular tissue matrix is made from a non-human mammalian tissue.

13. The composition of claim 12, wherein the non-human mammalian tissue is porcine tissue.

14. The composition of claim 12, wherein the non-human mammalian tissue is bovine tissue.

15. The composition of claim 1, further comprising one or more supplementary agents.

16. The composition of claim 15, wherein the one or more supplementary agents are selected from the group consisting of free radical scavengers, protein hydrolysates, tissue hydrolysates, and tissue breakdown products.

17. The composition of claim 15, wherein the supplementary agents are selected from the group consisting tocopherols, hyaluronic acid, chondroitin sulfate, and proteoglycans.

18. The composition of claim 15, wherein the one or more supplementary agents are selected from the group consisting of monosaccharides, disaccharides, oligosaccharides, polysaccharides, sugar alcohols, and starch derivatives.

19. The composition of claim 18, wherein the starch derivatives are selected from the group consisting of maltodextrins, hydroxyethyl starch (HES), and hydrogenated starch hydrolysates (HSH).

20. The composition of claim 18, wherein the sugar alcohols are selected from the group consisting of adonitol, erythritol, mannitol, sorbitol, xylitol, lactitol, isomalt, maltitol, and cyclitols.

21. The composition of claim 1, wherein the matrix is in non-particulate form.

22. The composition of claim 1, wherein the matrix is in particulate form.

23. A method of making a tissue matrix composition, the method comprising:
providing an acellular tissue matrix, the matrix being fully hydrated or partially dehydrated; and
a process comprising sequentially exposing the whole body of the matrix to increasing concentrations of a water-replacing reagent,
wherein the process: (i) results in a composition comprising a processed acellular tissue matrix that contains not more 30% of the water that the matrix contains if fully hydrated; and (ii) does not result in substantially irreversible shrinkage of the matrix.

24. The method of claim 23, further comprising, after the process, heating the composition at a temperature and for a period of time sufficient to inactivate substantially all viruses in the matrix.

25. The method of claim 24, wherein the composition is heated a temperature of 45° C. to 65° C. for more than 10 minutes.

26. The method of claim 23, further comprising, after the process, exposing the composition to γ, x, or e-beam radiation.

27. The method of claim 26, wherein the composition is exposed such that the matrix absorbs 6 kGy to 30 kGy of the radiation.

28. The method of claim 23, further comprising, after the process, exposing the composition to ultraviolet irradiation.

29. The method of claim 24, further comprising exposing the composition to γ, x, or e-beam radiation.

30. The method of claim 29, wherein the composition is exposed such that the matrix absorbs 6 to 30 kGy of the radiation.

31. The method of claim 24, further comprising exposing the composition to ultraviolet irradiation.

32. The method of claim 23, wherein the process comprises sequentially incubating the acellular matrix in at least two aqueous solutions, each solution containing a higher concentration of the water-replacing reagent than the previous solution in which the matrix was incubated.

33. The method of claim 23, wherein the process comprises exposing the matrix to a continuous increasing concentration gradient of the reagent.

34. The method of claim 23, wherein the water-replacing reagent comprises glycerol.

35. The method of claim 23, wherein the water-replacing reagent consists of glycerol.

36. The method of claim 23, wherein the water- replacing reagent comprises one or more water-replacing agents selected from the group consisting of DMSO and polyhydroxyl compounds.

37. The method of claim 23, wherein the polyhydroxyl compounds are selected from the group consisting of poly-glycerol, ethylene glycol, propylene glycol, polyethylene glycol (PEG), and polyvinyl alcohols (PVA).

38. The method of claim 37, wherein the water-replacing reagent comprises glycerol and ethylene glycol.

39. The method of claim 38, wherein the glycerol and the ethylene glycol are present in the reagent in equal concentrations by weight, by volume, or by molarity.

40. The method of claim 35, wherein the initial concentration of glycerol to which the matrix is exposed is about 40% volume to volume (v/v).

41. The method of claim 35, wherein the final concentration of glycerol is about 85% v/v.

42. The method of claim 32, wherein the water-replacing reagent comprises glycerol.

43. The method of claim 42, wherein the water-replacing reagent consists of glycerol.

44. The method of claim 43, wherein the at least two solutions are three solutions.

45. The method of claim 44, wherein the concentration of glycerol: (a) in the first solution is about 30% v/v; (b) in the second solution is about 60% v/v; and (c) in the third solution is about 85% v/v.

46. The method of claim 44, wherein the concentration of glycerol: (a) in the first solution is about 40% v/v; (b) in the second solution is about 60% v/v; and (c) in the third solution is about 85% v/v.

47. The method of claim 43, wherein the at least two solutions are four solutions.

48. The method of claim 47, wherein the concentration of glycerol: (a) in the first solution is about 40% v/v; (b) in the second solution is about 55% v/v; (c) in the third solution is about 70% v/v; and (d) in the fourth solution is about 85% v/v.

49. The method of claim 23, wherein the acellular matrix comprises dermis from which all, or substantially all viable cells have been removed.

50. The method of claim 23, wherein the acellular matrix comprises a tissue from which all, or substantially all, viable cells have been removed, wherein the tissue is selected from the group consisting of fascia, pericardial tissue, dura, umbilical cord tissue, placental tissue, cardiac valve tissue, ligament tissue, tendon tissue, arterial tissue, venous tissue, neural connective tissue, urinary bladder tissue, ureter tissue, and intestinal tissue.

51. The method of claim 23, wherein the matrix is made from human tissue.

52. The method of claim 23, wherein the matrix is made from non-human mammalian tissue.

53. The method of claim 52, wherein the non-human mammalian tissue is porcine tissue.

54. The method of claim 52, wherein the non-human mammalian tissue is bovine tissue.

55. The method of claim 23, wherein the matrix is non-particulate in form.

56. The method of claim 23, wherein the matrix is particulate in form.

57. The method of claim 23, wherein the water-replacing reagent comprises one or more supplementary agents.

58. The method of claim 57, wherein the one or more supplementary agents are selected from the group consisting of free radical scavengers, protein hydrolysates, tissue hydrolysates, and tissue breakdown products.

59. The method of claim 57, wherein the supplementary agents are selected from the group consisting tocophenols, hyaluronic acid, chondroitin sulfate, and proteoglycans.

60. The method of claim 57, wherein the one or more supplementary agents are selected from the group consisting of monosaccharides, disaccharides, oligosaccharides, polysaccharides, sugar alcohols, and starch derivatives.

61. The method of claim 60, wherein the starch derivatives are selected from the group consisting of maltodextrins, hydroxyethyl starch (HES), and hydrogenated starch hydrolysates (HSH)

62. The composition of claim 59, wherein the sugar alcohols are selected from the group consisting of adonitol, erythritol, mannitol, sorbitol, xylitol, lactitol, isomalt, maltitol and cyclitols.

63. A method of treatment, the method comprising:
(a) identifying a vertebrate subject as having an or organ, or tissue, in need of repair or amelioration; and
(b) placing the composition of claim 1 in or on the organ or tissue.

64. The method of claim 63, further comprising, prior to the placing, rinsing the composition in a physiological solution until the concentration of water-replacing agent in the composition is at a physiologically acceptable level.

65. The method of claim 63, wherein the vertebrate subject has an abdominal wall defect or an abdominal wall injury.

66. The method of claim 63, wherein the organ or tissue of the vertebrate subject is selected from the group consisting of skin, bone, cartilage, meniscus, dermis, myocardium, periosteum, artery, vein, stomach, small intestine, large intestine, diaphragm, tendon, ligament, neural tissue, striated muscle, smooth muscle, bladder, urethra, ureter, and gingiva.

67. The method of claim 63, wherein the organ or tissue of the vertebrate subject is abdominal wall fascia.

68. The method of claim 63, wherein the composition further comprises demineralized bone powder.

69. The method of claim 66, wherein the gingiva is, or is proximal to, receding gingiva.

70. The method of claim 66, wherein the gingiva comprises a dental extraction socket.

71. The method of claim 63, wherein the vertebrate subject is a mammal.

72. The method of claim 71, wherein the mammal is a human.

73. The method of claim 63, wherein the matrix is non-particulate in form.

74. The method of claim 63, wherein the matrix is particulate in form.
